SEQUENCE LISTING (50 sequences)

SEQ ID NO: 1 - protein, 861 aa, Piscine Myocarditis virus
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRYDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 2 - protein, 200 aa, synthetic construct
  REGION 1..200: note = Fragment of ORF-1 protein of Piscine Myocarditis virus
YDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSEGGTMRKRIPAIEMRENGVEGDLMNDDLYSIGTAAGYL

SEQ ID NO: 3 - protein, 50 aa, synthetic construct
  REGION 1..50: note = Fragment of ORF-1 protein of Piscine Myocarditis virus
EGGTMRKRIPAIEMRENGVEGDLMNDDLYSIGTAAGYLAVEGMAGAQGGI

SEQ ID NO: 4 - protein, 50 aa, synthetic construct
  REGION 1..50: note = Fragment of ORF-1 protein of Piscine Myocarditis virus
WDVVQYQLPGPDDEARGVMNTVGAMGGWTRAVTPVDNVATMRDNGVEGEP

SEQ ID NO: 5 - protein, 50 aa, synthetic construct
  REGION 1..50: note = Fragment of ORF-1 protein of Piscine Myocarditis virus
CGIVMSLPTSGTAVVDRLANFGLPPARAELREVPFGGYQRSVTNTNHRVK

SEQ ID NO: 6 - protein, 183 aa, Salmo Salar
MAVLKWLSICLTLFCQGTAASKPCRWTQFRLGKLNDVSIGLLSDMGGLFPLMCAEESVEQMFPEDLYKNTEGEDVYVVALEAMRYVEQLYNNSLTSVTWNKTKLNMFQNVIYRQVQNLELCVVGGVWESSGDGWSVTLKTYFNKLNTVLKEKEHSACAWEIVRKEIRENLVQFKKFIDSRVKL

SEQ ID NO: 7 - protein, 20 aa, synthetic construct
  REGION 1..20: note = Fragment of viral hemorrhagic septicemia virus G-protein (VHSV-G)
MEWNTFFLVILIIIIKSTTP

SEQ ID NO: 8 - protein, 21 aa, synthetic construct
  REGION 1..21: note = Fragment of viral hemorrhagic septicemia virus G-protein (VHSV-G)
VGGAFLLLVLCCCCKASPPIP

SEQ ID NO: 9 - protein, 779 aa, synthetic construct
  REGION 1..779: note = Fusion peptide
MEWNTFFLVILIIIIKSTTPQITQRPPVENISTYHADWDHHHHHHGGGSGGRMEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRGGGGSAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDIGGRNLIPSDWSFHWSIWPSLSGMGVVGGAFLLLVLCCCCKASPPIPNYGIPMQQFSRSQTV

SEQ ID NO: 10 - protein, 929 aa, synthetic construct
  REGION 1..929: note = Fusion peptide
MEWNTFFLVILIIIIKSTTPQITQRPPVENISTYHADWDHHHHHHGGGSGGRMEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRYDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSGGGGSWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDIGGRNLIPSDWSFHWSIWPSLSGMGVVGGAFLLLVLCCCCKASPPIPNYGIPMQQFSRSQTV

SEQ ID NO: 11 - protein, 666 aa, synthetic construct
  REGION 1..666: note = Piscine myocarditis virus ORF 1 with an internal deletion and a linker
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRGGGGSAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 12 - protein, 816 aa, synthetic construct
  REGION 1..816: note = Piscine myocarditis virus ORF 1 with an internal deletion and a linker
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRYDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSGGGGSWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 13 - protein, 162 aa, synthetic construct
  REGION 1..162: note = Piscine myocarditis virus fragment of ORF-1 protein
AVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 14 - protein, 10 aa, synthetic construct
  REGION 1..10: note = Piscine myocarditis virus fragment of ORF 1 protein
VSGGRAVVQK

SEQ ID NO: 15 - protein, 50 aa, synthetic construct
  REGION 1..50: note = Piscine myocarditis virus fragment of ORF 1 protein
VSVSGGRAVVQKGNKAEMNPVFVNRTPGQTTLGQPTTDTTGMTTADFLDI

SEQ ID NO: 16 - DNA, 2583 bp, Piscine myocarditis virus
  source 1..2583: mol_type = other DNA
atggaaccaaacacatctgtcattgcaacggagcagcagcaggctgccatgagagaggtggaggccgaggcggcggccagagacgaagtggtggagaagatcgcattcgctgaaggagcgatgatggtacagacgaggaggttaccatcaggaaagtcgtcggtaggaggttttctcggcgaactggcacagaacatacgtgccatgaatcggtcattgcacacagataccaacatgctgaccgaaggggcgatggtggacagagcgagggcaaaagtacacaaaatcattagggaagggaatttggactctagggtattttcaaacacggggagcaacactatgttgtcactgtgggtaccagcagtaccgggaccaccggcagtaccggagcattgggacgttgcgccgtcctggttcgtatgcagaccggggaaaaagggggggataaagatcacacaaagcgcatcaatggcagcattaaacccactatttagaggcgcagacgtggggccaatcgggacagcagtcagggcggatgtaaacgcattttcaatgaatgcagttctgggagcactaagagccgggggatttaacaccgaacattccctggtgtcattcgttgaaccactaattcggatcttgctaatgggggtacaaacacaagacagggggaccagcccatgggattgggttggagggatgagttcgcgaatagtcaatcccctagtattcacaacaagcgggaacttcttcccagggggaccaaatttgagggtgtggggagccaacgatacagtggccaggatagtaaacgttgaggactacatgcgcgaggcggccggggaggggaggttcgacgctggatggggaccggaattctggggtgggacaggggacgacgcagtggcggtggtaccgataagggcagtagaagcagggctaggagaagtaaacgcagggtggacattggcacacatggaatacccagtcaaggttagactacttgacgtcgacgaccgaacaattggaccaggggggagcctgcccctaaacgcaaacagagaatacacggcggcaggagctacgcatgtacccgggccctatgccagggtactgtacgtcgtcgtggaccaaaacgcagacaggtgtgtgggggtgagagtgcagggacagggtgctgtaattgacgtggacccggcgttgaattacgtgatagggggagcggatttggggatgttgccgttgatacagtggagtgtagggctgggggccgaggacatggcgcagggatcgattgcacagacgcagcgatgggtgaggatgtatggaaacgaggacgattgggaatcagcgtggcatctagtgtctagcgcgtacacagtgtacagcccggcattcaggagatcgggtgtcgcagtggagggaggattctgggcgcaaccagctgcaggggcagcaccgtttccactaggaggattggcagggtgggtgaggtacgacaatcaggcacgggcggcgcaggttgcactttgcagagagagggcggatatggcggagtgtccttggggggggtacagggagagaggggtgagaccggggagtgtggcaaactggcagtacgtaaggttcgatcccacagtggctgtaggagtagctgctcacttctggtcggtagtgaaggtgatggtggctcccgtcccagacagagcggctgctctggcggacatggcgtgggggaaggggaaggtgcaagccatgggtgaggatgtgatcaacgggcagatgggacaacctgagtccatgatgagaggggtggcgctgaacgagaaccagggactagcggcggctacagtcaggagggtggttgggctggagaacgagtcgatgcaaacaacgcactggagtacaacggaggtagcaatgaacgggtactacgggagagcaggagcaacagcacaccacgctgcatttccgttgtccgagggggggacaatgcgaaaacgaataccagctatagagatgagggagaacggggtggagggggacctgatgaacgatgatctctattcaattggaacggcagcggggtacctggcggtagaggggatggcaggtgcgcaggggggtatctgggacgtggtccagtaccagctgcctgggcctgacgatgaggcgaggggggtgatgaacacggtgggggcgatggggggatggacgagggcggtgacaccagtagacaatgtggccaccatgagggacaacggggttgagggggaaccttgtggaatagtgatgtctctaccaacaagtgggaccgctgtggtggataggttagctaatttcggattaccaccagcgagggcggaattaagagaagtaccatttggcgggtaccaaagatcagtcacaaacaccaaccacagagtcaaggtgagtgtgagtggggggcgagcagttgttcaaaaagggaacaaagccgagatgaatccagtctttgtcaataggacaccaggacaaacgaccctaggccaaccaacaacagacactacagggatgacaactgcagattttttagatata

SEQ ID NO: 17 - DNA, 552 bp, synthetic construct
  misc_feature 1..552: note = Nucleic acid sequence encoding IFNb with codon changes
  source 1..552: mol_type = other DNA
atggcagtactgaagtggctttccatctgcctgactctgttctgccaaggaaccgcggcctcaaagccttgtcggtggacccagttccggctggggaaactgaatgacgtttcaattggcctactctccgacatggggggactcttcccgctgatgtgcgccgaggagtcggtggagcagatgtttccagaggacctctacaagaacaccgagggcgaagatgtgtacgtggttgctctggaagccatgcgttacgtagagcagctgtacaataactcacttacctccgtcacctggaacaaaactaagctcaacatgttccagaacgttatctacagacaggtgcagaaccttgagctatgcgttgtaggcggggtttgggagtctagtggtgacggatggtccgttacacttaagacctactttaataaactgaacactgtgctgaaggagaaagagcatagcgcttgtgcctgggagattgtccgcaaggagattagggagaacctggttcagtttaagaagtttatcgactcaagagtgaaactgtga

SEQ ID NO: 18 - DNA, 552 bp, synthetic construct
  misc_feature 1..552: note = wild-type nucleic acid sequence encoding IFN-b protein
  source 1..552: mol_type = other DNA
atggctgtattgaaatggttgagcatttgcctgactctgttctgccaaggcacagcagcatcaaaaccttgcaggtggacgcagtttaggttggggaagctgaacgatgtgagcataggcctgctctcagatatgggtggactctttccacttatgtgtgcagaagaaagcgtcgaacaaatgtttccagaggatctttacaagaacacagagggtgaggacgtctatgtggtggcattggaggctatgcgatatgtggaacaattatacaacaacagtctgacgtctgtcacgtggaacaaaacaaaacttaacatgttccaaaacgtcatatatcgtcaagttcaaaacttagagttatgtgtcgtaggtggtgtttgggaatcctctggagatggatggtcggttactctgaaaacatacttcaacaagctgaacaccgtcttgaaagagaaggaacacagcgcatgcgcatgggagattgtgcgaaaggagattcgcgaaaacttggtgcagttcaagaaattcattgacagcagagtcaagctgtga

SEQ ID NO: 19 - DNA, 552 bp, synthetic construct
  misc_feature 1..552: note = Nucleic acid sequence encoding IFNb1
  source 1..552: mol_type = other DNA
atggctgtattgaaatggttgagcatttgcctgactctgttctgccaaggcacagtagcatcaaaaccttgcaggtggacgcagtttaggttggggaagctgaacgatgtgagcataggcctgctctcagatatgggtggactctttccacttatgtgtgcagaagaaaacgtcgaacaaatgtttccagaggatctttacaaaaacacagagggtgaggacgtctctgtggttgcattggaggctatgcgatatgtggaacaattatacaacaacagtctgacgtctgccacgtggagcaaaacaaaacttaacatgttccaaaacgtcatatatcgtcaagttcaaaacttagagttatgtgtcgtaggtggtgtttgggaatcctctggagatggatggtcggttactctgaaaacatacttcaacaagctgaacaccgtcttgaaagagaaggaatacagcgcatgcgcatgggagattgtgcgaaaggagattcgcgaaaacttggtgcagttcaagaaattcattgacagcagagtcaagctgtga

SEQ ID NO: 20 - DNA, 552 bp, synthetic construct
  misc_feature 1..552: note = Nucleic acid sequence encoding IFNb1 with codon changes
  source 1..552: mol_type = other DNA
atggctgtactgaaatggctgtccatctgtctgacactgttctgccagggaactgtagcttcaaagccatgcaggtggactcagttccgcttggggaaactaaatgacgtgagcatcggtttgctgtccgacatgggaggactgtttccactgatgtgtgctgaggagaacgtggagcagatgttccccgaggacctgtataagaacaccgagggcgaggacgtgtccgtcgtggccctggaggcgatgcggtacgtcgagcaactttacaacaactcgctcacttccgccacatggagtaagacgaagctcaacatgttccagaacgtgatctacagacaggtgcagaacctagagctgtgtgtggtggggggggtatgggagtccagtggggatggctggtccgttaccctgaagacatactttaacaagcttaataccgtgctgaaagagaaagaatactcggcgtgtgcctgggagattgtgcggaaggaaatcagggaaaatctggtgcagttcaagaaattcattgactcaagggtaaagttgtga

SEQ ID NO: 21 - protein, 114 aa, synthetic construct
  REGION 1..114: note = Fragment of sea lice gut protein
MENSRADVPNIEDKIPPKIEEDNELQGNSLTVPKSSNRESSNVRRMHTAVRLNEVIVNKSHDAKLVILNLPSPPKIMGPDKDASYMEFLEVLTEGLERVLMVRGGGREVITIYS

SEQ ID NO: 22 - protein, 50 aa, synthetic construct
  REGION 1..50: note = Fragment of Piscine myocarditis virus ORF-1 protein
CRERADMAECPWGGYRERGVRPGSVANWQYVRFDPTVAVGVAAHFWSVVK

SEQ ID NO: 23 - protein, 50 aa, synthetic construct
  REGION 1..50: note = Fragment of Piscine myocarditis virus ORF-1 protein
VMVAPVPDRAAALADMAWGKGKVQAMGEDVINGQMGQPESMMRGVALNEN

SEQ ID NO: 24 - protein, 50 aa, synthetic construct
  REGION 1..50: note = Fragment of Piscine myocarditis virus ORF-1 protein
QGLAAATVRRVVGLENESMQTTHWSTTEVAMNGYYGRAGATAHHAAFPLS

SEQ ID NO: 25 - protein, 100 aa, synthetic construct
  REGION 1..100: note = Fragment of Piscine myocarditis virus ORF-1 protein
QGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGI

SEQ ID NO: 26 - protein, 100 aa, synthetic construct
  REGION 1..100: note = Fragment of Piscine myocarditis virus ORF-1 protein
EGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEP

SEQ ID NO: 27 - protein, 100 aa, synthetic construct
  REGION 1..100: note = Fragment of Piscine myocarditis virus ORF-1 protein
WDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVK

SEQ ID NO: 28 - protein, 100 aa, synthetic construct
  REGION 1..100: note = Fragment of Piscine myocarditis virus ORF-1 protein
CGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 29 - protein, 150 aa, synthetic construct
  REGION 1..150: note = Fragment of Piscine myocarditis virus ORF-1 protein
QGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEP

SEQ ID NO: 30 - protein, 150 aa, synthetic construct
  REGION 1..150: note = Fragment of Piscine myocarditis virus ORF-1 protein
EGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVK

SEQ ID NO: 31 - protein, 150 aa, synthetic construct
  REGION 1..150: note = Fragment of Piscine myocarditis virus ORF-1 protein
WDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 32 - protein, 200 aa, synthetic construct
  REGION 1..200: note = Fragment of Piscine myocarditis virus ORF-1 protein
QGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVK

SEQ ID NO: 33 - protein, 200 aa, synthetic construct
  REGION 1..200: note = Fragment of Piscine myocarditis virus ORF-1 protein
EGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 34 - protein, 399 aa, synthetic construct
  REGION 1..399: note = Fragment of Piscine myocarditis virus ORF-1 protein
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVD

SEQ ID NO: 35 - protein, 499 aa, synthetic construct
  REGION 1..499: note = Fragment of piscine myocarditis virus Orf-1 protein
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVR

SEQ ID NO: 36 - protein, 5 aa, synthetic construct
  REGION 1..5: note = Linker amino acid sequence
GGGGS

SEQ ID NO: 37 - DNA, 15 bp, synthetic construct
  misc_feature 1..15: note = Nucleic acid sequence encoding the linker
  source 1..15: mol_type = other DNA
ggtggcggtggctcg

SEQ ID NO: 38 - protein, 30 aa, synthetic construct
  REGION 1..30: note = Fragment of fusion protein of Atlantic Salmon Paramyxovirus
MDGPKFRFVLLILLTAPARGQVDYDKLLKV

SEQ ID NO: 39 - protein, 77 aa, synthetic construct
  REGION 1..77: note = Fragment of fusion protein of Atlantic Salmon Paramyxovirus
LAKSEKILSGINPNIINTEMVLVAVILSLVCAMVVIGIVCWLSILTKWVRSCRADCRRPNKGPDLGPIMSSQDNLSF

SEQ ID NO: 40 - protein, 26 aa, synthetic construct
  REGION 1..26: note = Fragment of Hemagglutinin (HE) protein of Infectious salmon anemia virus (ISAV)
MARFIILFLLLAPVYSRLCLRNYPDT

SEQ ID NO: 41 - protein, 61 aa, synthetic construct
  REGION 1..61: note = Fragment of Hemagglutinin (HE) protein of Infectious salmon anemia virus (ISAV)
TFNTNQVEQPANSVLSNIFISMGVAGFGIALFLAGWKACIWIAAFMYKSRGRIPPSNLSVA

SEQ ID NO: 42 - DNA, 24 bp, synthetic construct
  misc_feature 1..24: note = MX forward primer
  source 1..24: mol_type = other DNA
gatgctgcacctcaagtcctatta

SEQ ID NO: 43 - DNA, 21 bp, synthetic construct
  misc_feature 1..21: note = MX reverse primer
  source 1..21: mol_type = other DNA
cggatcaccatgggaatctga

SEQ ID NO: 44 - DNA, 20 bp, synthetic construct
  misc_feature 1..20: note = MX assay probe
  source 1..20: mol_type = other DNA
caggatatccagtcaacgtt

SEQ ID NO: 45 - DNA, 2583 bp, synthetic construct
  misc_feature 1..2583: note = Codon optimized nucleic acid sequence encoding Piscine myocarditis virus ORF 1 protein
  source 1..2583: mol_type = other DNA
atggagcccaacaccagcgtcattgccacagagcagcagcaggctgcaatgagggaggtggaggccgaggccgccgctagagacgaggtggtcgagaaaatcgcatttgcagagggtgcaatgatggtgcagactcgtaggctgcccagcggcaagtcatccgtcggtggatttctcggggagctcgcccagaacattagggcaatgaacaggtccctccatacagacactaatatgctcaccgagggcgccatggtcgacagagctagagctaaagtccacaagattatcagggagggtaacctcgattcaagagtcttcagcaacacaggatcaaacaccatgctcagtctgtgggtgcccgctgtccccggtccacccgctgtgcccgagcattgggatgtcgctcccagctggtttgtctgccggccaggaaagaaaggtggaattaagatcactcagtcagcatctatggccgccctgaatcctctgtttagaggagccgatgtcggcccaattggtacagccgtgcgtgcagacgtcaacgcattttccatgaatgccgtgctgggggcactgagggccggaggtttcaatacagagcacagtctggtctcatttgtcgagccactgattagaatcctgctgatgggagtgcagacacaggacagaggaacatcaccttgggactgggtgggcggaatgagttcccgtattgtgaatcccctggtctttacaactagtggaaatttctttcccggcggacccaacctcagagtgtggggtgctaacgataccgtcgcccgtattgtcaacgtcgaggactacatgcgcgaggccgcaggcgagggaagatttgacgccggatggggtccagagttttggggtggaactggggacgatgcagtcgccgtcgtgcctattagggccgtggaggctgggctcggagaggtcaacgccggttggaccctcgcccacatggagtaccccgtcaaagtccggctcctggacgtcgacgacaggaccatcggccccggagggagtctgccactcaacgcaaatcgggagtacaccgctgccggagctacacacgtgccaggaccatacgccagggtgctgtacgtcgtggtcgatcagaacgccgacaggtgcgtgggtgtcagggtccaggggcagggggcagtcatcgacgtcgatcccgccctgaactacgtgatcgggggagccgacctgggtatgctccccctgatccagtggagtgtgggcctgggagccgaggacatggctcagggtagcattgcccagacccagcgttgggtcagaatgtatgggaacgaggacgattgggagagtgcctggcatctggtctcaagtgcctatactgtctatagtcctgcttttcgccggagtggcgtggctgtcgagggaggcttctgggcacagcccgctgccggtgcagctccatttcccctgggcgggctcgcaggatgggtgaggtatgataaccaggccagggccgctcaggtggccctgtgcagggagcgggctgacatggcagagtgcccctggggaggctatcgggagaggggagtgaggcccggtagtgtcgccaactggcagtacgtgcgctttgaccccaccgtggctgtcggagtggctgcccacttctggagtgtcgtgaaagtcatggtcgcacccgtgccagatcgtgctgcagctctggccgatatggcctggggaaagggaaaagtccaggccatgggggaggacgtcatcaatggccagatgggacagccagagagtatgatgcgcggagtcgccctgaacgagaaccaggggctggcagccgctactgtgaggcgtgtcgtcggactggagaatgagagcatgcagacaactcactggtcaaccacagaggtggctatgaatggttactatggcagggccggtgccacagcacaccatgctgccttccctctctcagagggaggtacaatgaggaagcgtatccctgcaatcgagatgcgtgagaacggtgtggagggggacctgatgaacgatgacctctacagcatcgggactgccgccggatatctggctgtggagggcatggccggcgctcagggcggaatttgggacgtggtgcagtatcagctgcctggacccgatgacgaggctcgtggtgtgatgaatactgtgggagccatgggtggatggaccagagcagtgactcccgtggacaacgtggctactatgcgtgacaatggagtggagggcgagccttgcggtatcgtcatgtccctgccaacttccggcaccgctgtggtcgaccggctggccaactttggcctcccaccagcaagggctgagctcagggaggtcccattcggaggataccagcgcagcgtgacaaacactaaccacagggtgaaggtctccgtctctggaggcagagcagtcgtgcagaagggaaacaaggcagagatgaatcccgtgttcgtcaacaggacacccggccagacaacactgggacagcccaccacagacactacaggcatgaccactgccgacttcctcgacatt

SEQ ID NO: 46 - protein, 856 aa, synthetic construct
  REGION 1..856: note = Piscine myocarditis virus ORF 1 fragment
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRYDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSGGGGSGNKAEMNPVFVNRTPGQTTLGQPTTDTTGMTTADFLDI

SEQ ID NO: 47 - protein, 969 aa, synthetic construct
  REGION 1..969: note = Fusion protein comprising a fragment of ORF-1 protein of Piscine Myocarditis virus
MEWNTFFLVILIIIIKSTTPQITQRPPVENISTYHADWDHHHHHHGGGSGGRMEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRYDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSGGGGSGNKAEMNPVFVNRTPGQTTLGQPTTDTTGMTTADFLDIGGRNLIPSDWSFHWSIWPSLSGMGVVGGAFLLLVLCCCCKASPPIPNYGIPMQQFSRSQTV

SEQ ID NO: 48 - protein, 39 aa, synthetic construct
  REGION 1..39: note = Fragment of viral hemorrhagic septicemia virus G-protein (VHSV-G)
MEWNTFFLVILIIIIKSTTPQITQRPPVENISTYHADWD

SEQ ID NO: 49 - protein, 58 aa, synthetic construct
  REGION 1..58: note = Fragment of viral hemorrhagic septicemia virus G-protein (VHSV-G)
NLIPSDWSFHWSIWPSLSGMGVVGGAFLLLVLCCCCKASPPIPNYGIPMQQFSRSQTV

SEQ ID NO: 50 - protein, 183 aa, Salmo Salar
MAVLKWLSICLTLFCQGTVASKPCRWTQFRLGKLNDVSIGLLSDMGGLFPLMCAEENVEQMFPEDLYKNTEGEDVSVVALEAMRYVEQLYNNSLTSATWSKTKLNMFQNVIYRQVQNLELCVVGGVWESSGDGWSVTLKTYFNKLNTVLKEKEYSACAWEIVRKEIRENLVQFKKFIDSRVKL

(19) United States
(12) Patent Application Publication
(10) Pub. No.: US 2025/0057937 A1
(43) Pub. Date: Feb. 20, 2025

(54) COMPOSITIONS AND METHODS FOR EXPRESSING ANTIGENS ON CELL MEMBRANE

(71) Applicant: Zoetis Services LLC, Parsippany, NJ (US)

(72) Inventors: Oyvind Haugland, Oslo (NO); Marius Andre de Feijter Karlsen, Oslo (NO); Linda Helena Hauge, Oslo (NO); Marit Rode, Oslo (NO)

(21) Appl. No.: 18/721,713

(22) PCT Filed: Dec. 19, 2022
(86) PCT No.: PCT/US2022/081912

§ 371 Date: Jun. 19, 2024

Related U.S. Application Data
(60) Provisional application No. 63/291,565, filed on Dec. 20, 2021.

(51) Int. Cl. A61K 39/15; A61K 39/00; A61P 31/14; C07K 14/005; C12N 15/63

(52) U.S. Cl. CPC A61K 39/15; A61P 31/14; C07K 14/005; C12N 15/63; A61K 2039/53; A61K 2039/552; C07K 2319/02; C07K 2319/03; C12N 2720/12222; C12N 2720/12234; C12N 2760/20022

(57) ABSTRACT

Disclosed is a system for targeting expression of antigens delivered via DNA vaccines to cell surface. Antigens, vaccines, and methods of using of these vaccines in prevention of fish from infections are also disclosed.

### FIELD OF THE INVENTION

This invention is generally in the field of vaccines.

### BACKGROUND

Aquaculture has experienced an enormous growth in productive terms, accounting to >527% in the 1990-2018 time frame. In 2018, aquaculture contributed to approximately 46% of the global total production of aquatic organisms (179 M tons) and 52% of seafood for human consumption (fish, crustaceans, mollusks, and other aquatic animals, excluding aquatic mammals, reptiles, seaweeds, and other aquatic plants).

Commercial aquaculture is impacted by infectious diseases caused primarily by bacteria, viruses, parasites, and, to a lesser extent, fungi. Bacterial diseases can inflict significant biological, thus economic losses. While these are usually controllable with antibiotics, the indiscriminate use of these pharmaceuticals is ultimately a threat to human health because of the development and transfer of resistance mechanisms among bacterial species, some of which are also human pathogens. In addition, some of these pathogens are difficult to grow in culture. Accordingly, modified live or inactivated vaccines that protect against these pathogens are not commercially viable.

DNA vaccines have become an attractive approach for generating antigen-specific immune responses because of their stability and simplicity of delivery. DNA vaccines can be easily prepared in large scale with high purity, repeatedly administered and are highly stable relative to proteins and other biological polymers.

Among the many forms of nucleic acid vaccines that can be constructed, circular DNA plasmids are the simplest. DNA vaccination involves immunization with a circular DNA plasmid that contains the gene (or genes) that code for an antigen. Indeed, injection of free DNA (naked DNA) stimulates effective and long-time immune responses to the protein (antigen) encoded by the gene vaccine. When plasmid DNA is injected into an individual, the plasmid is taken up by cells and its genetic information is translated into the immunizing protein. This enables the host immune system to respond to the antigen.

However, DNA vaccines are relatively new and need exists in improving the efficiency of these vaccines.

### SUMMARY OF INVENTION

In a first aspect, the invention provides fusion protein comprising, from N-terminus to C-terminus:
a) N-terminal secretion signal sequence of a secreted or a first membrane-bound protein;
b) an antigen from a non-enveloped virus;
c) a transmembrane domain of a second membrane-bound protein.

In certain embodiments, the first membrane-bound protein is identical to the second membrane-bound protein. In certain embodiments, said membrane bound protein is viral hemorrhagic septicemia virus G-protein (VHSV-G). Preferably, said N-terminal secretion signal sequence is at least 90% identical to SEQ ID NO: 7 or SEQ ID NO: 48, or wherein said secretion signal sequence comprises a portion of SEQ ID NO: 48 or a sequence at least 90% identical to SEQ ID NO: 48, wherein said portion comprises SEQ ID NO: 7 or a sequence that is at least 90% identical to SEQ ID NO: 7. In some embodiments, said N-terminal secretion signal sequence is at least 90% identical to SEQ ID NO: 7 and at least half of differing amino acids in said N-terminal secretion signal are conservative substitutions.

In certain embodiments, said transmembrane domain is at least 90% identical to SEQ ID NO: 8 or SEQ ID NO: 49, or wherein said transmembrane fragment comprises a portion of SEQ ID NO: 49 or a sequence at least 90% identical to SEQ ID NO: 49, wherein said portion comprises SEQ ID NO: 8 or a sequence that is at least 90% identical to SEQ ID NO: 8. Preferably, said transmembrane domain is at least 90% identical to SEQ ID NO: 8 and at least half of differing amino acids in said N-terminal secretion signal are conservative substitutions.

In certain embodiments, the non-enveloped virus is a non-enveloped virus affecting fish that may be selected from the group consisting of Piscine Myocarditis Virus (PMCV), Piscine Orthoreovirus (PRV), Infectious Pancreatic Necrosis Virus (IPNV), and betanodavirus.

In certain embodiments, the antigen is encoded by ORF-1 of piscine myocarditis virus (PMCV). In some embodiments, the antigen is a protein comprising, from N- to C- terminus:
a. a sequence that is at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID NO: 34 or SEQ ID NO: 35, and
b. an amino acid sequence that is at least 95%, 96%, 97%, 98%, 99%, or 100% identical to any one of SEQ ID NOs: 25-33, wherein,
i) said protein lacks SEQ ID NO: 15 or a sequence at least 90% identical to SEQ ID NO: 15; and/or
ii) compared to SEQ ID NO: 1, said protein comprises an internal deletion that is at least four consecutive amino acids long.

In certain embodiments, the protein is at least 90% identical to SEQ ID NO: 11 or SEQ ID NO: 12.

In certain embodiments, the protein that is at least 95% identical to SEQ ID NO: 25 and lacks SEQ ID NO: 14 or a sequence that is at least 90% identical to SEQ ID NO: 14. Preferably, the protein lacks one or more of SEQ ID NO: 4, SEQ ID NO: 5, SEQ ID NO: 15 or a sequence that is at least 90% identical thereto.

In certain other embodiments, the protein of the invention includes the sequence that is at least 95% identical to SEQ ID NO: 26 and lacks SEQ ID NO: 14 or a sequence that is at least 90% identical to SEQ ID NO: 14. The protein may include the sequence that is at least 95% identical to SEQ ID NO: 26 and lacks SEQ ID NO: 14 or a sequence that is at least 90% identical to SEQ ID NO: 14 and further lack one or more of SEQ ID NO: 5, 15, or 24 or a sequence that it at least 90% identical thereto.

In other embodiments, the protein includes the sequence that is at least 95% identical to SEQ ID NO: 27, and lacks SEQ ID NO: 14 or a sequence that is at least 90% identical to SEQ ID NO: 14. The protein may include the sequence that is at least 95% identical to SEQ ID NO: 27 and lacks SEQ ID NO: 14 or a sequence that is at least 90% identical to SEQ ID NO: 14 and further lack one or more of SEQ ID NO: 3, 15, or 24 or a sequence that it at least 90% identical thereto.

In other embodiments, the protein includes the sequence that is at least 90% identical to SEQ ID NO: 28. Optionally, the protein lacks one or more of SEQ ID NOs 3, 4, or 24 or a sequence that it at least 90% identical thereto.

In other embodiments, the protein comprises SEQ ID NO: 29 or SEQ ID NO: 30 or a sequence at least 95%, or 96%, or 97% or 98% or 99% identical to any one of these sequences. Preferably, the protein lacks at least one of SEQ ID NO: 3 or SEQ ID NO: 24, or an amino acid sequence that is at least 90% identical to SEQ ID NO: 3 or SEQ ID NO: 24.

In other embodiments, the protein comprises SEQ ID NO: 32 or a sequence at least 95%, or 96%, or 97% or 98% or 99% identical thereto and, optionally, lacks at least one of SEQ ID NO: 14 or SEQ ID NO: 15, or an amino acid sequence that is at least 90% identical to SEQ ID NO: 14 or SEQ ID NO: 15.

In other embodiments, the protein comprises SEQ ID NO: 33 or a sequence at least 95%, or 96%, or 97% or 98% or 99% identical thereto and optionally, lacks SEQ ID NO: 24 an amino acid sequence that is at least 90% identical to SEQ ID NO: 24.

In any of the embodiments described above, the protein may further optionally lack at least one of SEQ ID NOs 2 and 13, or an amino acid sequence that is at least 90% identical to SEQ ID NO: 2 or 13.

In certain embodiments, the protein comprises SEQ ID NO: 11 or 12 or a sequence that is at least 90%, or at least 91%, or at least 92%, or at least 93%, or at least 94%, or at least 95%, or at least 96%, or at least 97%, or at least 98%, or at least 99%, or 100% identical to SEQ ID NO: 11 or SEQ ID NO: 12.

In certain embodiments, the protein includes SEQ ID NO: 15 or a sequence at least 90% identical thereto and any one of SEQ ID NOs 4, 5, 25, 26 27, 29, 30, 32, (or an amino acid sequence that is at least 90% identical to SEQ ID NO: 4, 5, 25, 26 27, 29, 30, or 32) wherein, compared to SEQ ID NO: 1, said protein comprises an internal deletion that is at least four consecutive amino acids long, and, optionally, wherein said protein comprises a linker in place of said internal deletion.

In yet other embodiments, the protein lacks SEQ ID NO: 13 or a sequence that is at least 90% identical to SEQ ID NO: 13 and contains SEQ ID NO: 25 or a sequence at least 95%, or 96%, or 97% or 98% or 99% identical to SEQ ID NO: 25.

In the second aspect, the invention provides a nucleic acid sequence that encodes the fusion protein according any of the embodiments of the first aspect of the invention.

In the third aspect, the invention provides a cassette comprising the nucleic acid sequence according to any embodiment of the second aspect.

In the fourth aspect of the invention, provided is a vector comprising the cassette according to any embodiment of the third aspect of the invention.

In certain embodiments, the vector also encodes a nucleic acid sequence that encodes an immunomodulator. In certain embodiments, the immunomodulator is interferon, such as IFNb type interferon that comprises SEQ ID NO: 6, wherein said nucleic acid sequence that encodes interferon is 65-90% identical to SEQ ID NO: 18 or 65-90% identical to SEQ ID NO: 19, and wherein codon frequency is preserved in said nucleic acid sequence. In certain embodiments, the nucleic acid sequence that encodes interferon comprises SEQ ID NO: 17 or SEQ ID NO: 20 or a nucleic acid sequence are at least 90% identical to SEQ ID NO: 17 or SEQ ID NO: 20, wherein said nucleic acid sequence that encodes interferon encodes SEQ ID NO: 6 or an amino acid sequence that is 95% identical to SEQ ID NO: 6.

In certain embodiments, the vector may be a plasmid vector or a viral vector.

In the fifth aspect, the disclosure provides a vaccine comprising the fusion protein according any embodiment of the first aspect or the vector according to the fourth aspect of the invention. In certain embodiments, the vaccine further comprises an adjuvant. In certain embodiments, the vaccine also provides a carrier. In one embodiment, the carrier is a lipid or a liposomal carrier.

In certain embodiments, the vaccine is a polyvalent vaccine and comprises one or more additional antigens, preferably selected from the groups consisting of SAV (salmonid alphavirus, including SAV-1, SAV-2, SAV-3, SAV-4, SAV-5, and SAV-6), ISAV (infectious salmon anemia virus), IPNV (infectious pancreatic necrosis virus), ASPV (Atlantic salmon poxvirus), IHNV (Infectious hematopoietic necrosis virus), VHSV (Viral hemorrhagic septicemia virus), PRV (piscine orthoreovirus), Aeromonas salmonicida subs. Salmonicida, Vibrio (Listonella) anguillarum serotype O1, Vibrio (Listonella) anguillarum serotype O2a, Vibrio salmonicida, Moritella viscosa, and sea lice proteins.

In the sixth aspect, the invention provides a method of protecting a salmonid against PMCV infection, the method comprising administering to the salmonid in need thereof the vaccine according to any embodiment of the fifth aspect of the invention. In certain embodiments said salmonid weighs between 15 and 200 grams. In certain embodiments, the salmonid is Atlantic salmon (Salmo salar), rainbow trout (Oncorhynchus mykiss), Coho Salmon (Oncorhynchus kisutch) or Chinook salmon (Oncorhynchus tshawytscha).

### BRIEF DESCRIPTION OF THE DRAWINGS

FIG. 1A is a general virion structure of Totiviridae. FIG. 1B is an illustration of genome organization of PMCV. The genome includes three large ORFs.

FIG. 2A in an illustration of the strategy for routing vaccine antigen for expression on the cell-surface. FIG. 2B is an illustration of the strategy to facilitate secreted antigen

FIG. 3A is a photograph demonstrating detection of PMCV ORF1 in non-permeabilized cells. FIG. 3B demonstrates the results obtained with different expression cassettes used for routing full length PMCV ORF-1 to the cell surface.

FIG. 4A is an evaluation of fluorescence intensity of the truncated capsid variants by IF-staining of transfected CHH-1 cells. FIG. 4B is an illustration of protein expression by Western blotting detection from the membrane-fraction by immunoprecipitation (IP-Western). The two blots represent two different experiments.

FIG. 5 is an Evaluation of protein expression in five different plasmid backbones by immunoprecipitation from total cell lysate of transfected CHH-1 cells and Western blotting.

### DETAILED DESCRIPTION

In order to better explain the invention, the following definitions are provided.

The term “about” as applied to a reference number refers to the reference number plus or minus 10 percent of said value.

The term “codon frequency is preserved in said nucleic acid sequence” describes a nucleic acid sequence wherein the frequencies of at least 50% of codons in the nucleic acid sequence of the invention do not deviate by more than 40% from the frequencies of the same codons in the genome of the host. When, for example, the frequency of a given codon in the host genome is, say, 20%, then, this codon is counted among the at least 50% of codons whose frequency does not deviate by more than 40% from the frequency of the same codon in the genome of the host if the frequency of the same codon in the nucleic acid sequence according to the invention is between 12% (40% less than 20%) and 28% (40% more than 20%).

For example, codon frequencies in the genome of Atlantic Salmon (Salmo salar) are provided in Table 1.

TABLE 1
CODON FREQUENCY IN THE GENOME OF ATLANTIC SALMON

| AMINO ACID | CODON | Per 1000 | FREQUENCY |
|---|---|---|---|
| GLY | GGG | 15.87 | 21.91 |
| GLY | GGA | 20.24 | 27.94 |
| GLY | GGT | 14.03 | 19.37 |
| GLY | GGC | 22.3 | 30.78 |
| ASP | GAT | 16.76 | 32.69 |
| ASP | GAC | 34.51 | 67.31 |
| GLU | GAG | 45.67 | 76.69 |
| GLU | GAA | 13.88 | 23.31 |
| VAL | GTG | 29.06 | 45.71 |
| VAL | GTA | 6.19 | 9.74 |
| VAL | GTT | 9.77 | 15.37 |
| VAL | GTC | 18.56 | 29.19 |
| ALA | GCG | 7.2 | 11.08 |
| ALA | GCA | 13.42 | 20.65 |
| ALA | GCT | 17.5 | 26.92 |
| ALA | GCC | 26.88 | 41.35 |
| ARG | AGG | 13.4 | 26.85 |
| ARG | AGA | 10.12 | 20.28 |
| ARG | CGG | 6.61 | 13.25 |
| ARG | CGA | 4.01 | 8.036 |
| ARG | CGT | 5.74 | 11.50 |
| ARG | CGC | 10.02 | 20.08 |
| SER | AGT | 11.2 | 13.54 |
| SER | AGC | 21.01 | 25.40 |
| SER | TCG | 5.31 | 6.42 |
| SER | TCA | 10.15 | 12.27 |
| SER | TCT | 13.86 | 16.76 |
| SER | TCC | 21.18 | 25.61 |
| LYS | AAG | 39.68 | 69.87 |
| LYS | AAA | 17.11 | 30.13 |
| ASN | AAT | 11.42 | 28.36 |
| ASN | AAC | 28.85 | 71.64 |
| MET | ATG | 27.27 | 100 |
| ILE | ATA | 6.37 | 13.59 |
| ILE | ATT | 11.34 | 24.19 |
| ILE | ATC | 29.17 | 62.22 |
| TRP | TGG | 11.53 | 100 |
| CYS | TGT | 11.05 | 46.80 |
| CYS | TGC | 12.56 | 53.20 |
| TYR | TAT | 8.12 | 27.21 |
| TYR | TAC | 21.72 | 72.79 |
| LEU | TTG | 10.16 | 10.97 |
| LEU | TTA | 3.44 | 3.71 |
| LEU | CTG | 45.99 | 49.65 |
| LEU | CTA | 7.18 | 7.75 |
| LEU | CTT | 7.87 | 8.50 |
| LEU | CTC | 17.98 | 19.41 |
| PHE | TTT | 12.55 | 32.54 |
| PHE | TTC | 26.02 | 67.46 |
| GLN | CAG | 35.96 | 81.21 |
| GLN | CAA | 8.32 | 18.79 |
| HIS | CAT | 8.63 | 32.43 |
| HIS | CAC | 17.98 | 67.57 |
| PRO | CCG | 5.93 | 11.09 |
| PRO | CCA | 13.3 | 24.86 |
| PRO | CCT | 13.89 | 25.97 |
| PRO | CCC | 20.37 | 38.08 |
| THR | ACG | 6.91 | 11.85 |
| THR | ACA | 14.69 | 25.18 |
| THR | ACT | 12.76 | 21.88 |
| THR | ACC | 23.97 | 41.09 |
| AMINO ACID | CODON | Per 1000 | FREQUENCY |

Codon CGC encodes arginine with frequency 20.08%—i.e., 20.08% arginine residues in the genome of Salmo salar are encoded by CGC. If in a nucleic acid sequence between 12.048% and 28.112% of arginine residues are encoded by CGC, then codon CGC is counted within among the at least 50% of codons whose frequency does not deviate by more than 40% from the frequency of the same codon in the genome of Salmo salar.

“Therapeutically effective amount” refers to an amount of an antigen or vaccine that would induce an immune response in a subject receiving the antigen or vaccine which is adequate to prevent or reduce signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response of an animal to a vaccine may be evaluated, e.g., indirectly through measurement of antibody titers, lymphocyte proliferation assays, or directly through monitoring signs and symptoms after challenge with wild type strain. The protective immunity conferred by a vaccine can be evaluated by measuring, e.g., reduction in clinical signs such as mortality, morbidity, temperature number, overall physical condition, and overall health and performance of the subject. The amount of a vaccine that is therapeutically effective may vary depending on the particular adjuvant used, the particular antigen used, or the condition of the subject, and can be determined by one skilled in the art.

“Treating” refers to preventing a disorder, condition, or disease to which such term applies, or to preventing or reducing one or more symptoms of such disorder, condition, or disease.

The invention provides a platform allowing an improved targeting of an antigen to cell surface. Thus, such engineered antigens will localize to cell surface where they can be recognized by the host's immune system and thus protective immune response would be generated. Thus, in a first aspect, the invention provides a fusion protein comprising, from N-terminus to C-terminus:
a) N-terminal secretion signal sequence of a secreted or a first membrane-bound protein;
b) the antigen;
c) a transmembrane domain of a second membrane-bound protein.

In certain embodiments, said first protein may be selected from the group consisting of Interferon a, Interferon b, Interferon c, Interferon d, Interferon gamma, interleukin-2, interleukin-4, interleukin-12. The second protein may be independently selected from the fusion protein of Atlantic Salmon Paramyxovirus, Hemagglutinin (HE) protein of Infectious salmon anemia virus (ISAV), or G-protein of viral hemorrhagic septicemia virus.

The first and the second protein may be identical or different from each other. In some embodiments, the first and/or the second protein may be the fusion protein of Atlantic Salmon Paramyxovirus whose signaling and transmembrane domain sequences comprise SEQ ID NOs: 38 and 39, respectively. In some embodiments, the first and the second protein may be the Hemagglutinin (HE) protein of Infectious salmon anemia virus (ISAV) whose secretion signaling and transmembrane domain sequences comprise SEQ ID NOs: 40 and 41, respectively. In other embodiments, the first and the second proteins can be different. For example, the first protein may comprise SEQ ID NO: 38 and the second protein may comprise SEQ ID NO: 41. In other embodiments, the first protein may comprise SEQ ID NO: 40 and the second protein may comprise SEQ ID NO: 39. In certain embodiments, the first protein is selected from the group consisting of SEQ ID NOs 7, 38, and 40, and the second protein is independently selected from the group consisting of SEQ ID NOs 8, 39, and 41, or sequences that are at least 90% identical to the respective reference sequences, wherein, preferably, the differing amino acids are conservative substitutions.

In some embodiments, the first membrane protein is identical to the second membrane protein and is viral hemorrhagic septicemia virus G-protein (VHSV-G).

Thus, in some embodiments, said N-terminal secretion signal sequence is at least 90%, or at least 91%, or at least 92%, or at least 93%, or at least 94%, or at least 95%, or at least 96%, or at least 97%, or at least 98%, or at least 99%, or 100% identical to SEQ ID NO: 7.

As described above, preferably, the mutations leading to the differences from SEQ ID NO: 7 are substitutions. In certain advantageous embodiments, at least half (or at least 60% or at least 70% or at least 80%, or at least 90%) of differing amino acids in said N-terminal secretion signal are conservative substitutions.

In the embodiments, when VHSV-G protein is the source of both the N-terminal secretion signal and the transmembrane domain, said transmembrane domain is at least 90%, or at least 91%, or at least 92%, or at least 93%, or at least 94%, or at least 95%, or at least 96%, or at least 97%, or at least 98%, or at least 99%, or 100% identical to SEQ ID NO: 8.

As described above, preferably, the mutations leading to the differences from SEQ ID NOs 7 and 8 are substitutions. In certain advantageous embodiments, at least half (or at least 60% or at least 70% or at least 80%, or at least 90%) of differing amino acids in said transmembrane domain are conservative substitutions.

Additional fragments of the first and/or second protein may also be included. Thus, in certain embodiments, SEQ ID NO: 7 or a protein that is at least 90% identical to SEQ ID NO: 7 may be included within SEQ ID NO: 48 or an amino acid sequence that is at least 90% identical to SEQ ID NO: 48 or a fragment thereof. It should be understood that said fragment would include SEQ ID NO: 7 or a sequence that is at least 90% identical to SEQ ID NO: 7.

Independently, SEQ ID NO: 8 or a protein that is at least 90% identical to SEQ ID NO: 8 may be included within SEQ ID NO: 49 or an amino acid sequence that is at least 90% identical to SEQ ID NO: 49 or a fragment thereof. It should be understood that said fragment would include SEQ ID NO: 8 or a sequence that is at least 90% identical to SEQ ID NO: 8.

Similarly to SEQ ID NOs 7 and 8, the mutations leading to the differences from SEQ ID NOs 48 and 49 are substitutions. In certain advantageous embodiments, at least half (or at least 60% or at least 70% or at least 80%, or at least 90%) of differing amino acids in said N-terminal secretion signaling sequence and/or in the transmembrane domain are conservative substitutions.

In certain embodiments, the non-enveloped virus is a non-enveloped virus affecting fish. Suitable examples of such non-enveloped viruses affecting fish include, without limitations, Piscine Myocarditis Virus (PMCV), Piscine Orthoreovirus (PRV), Infectious Pancreatic Necrosis Virus (IPNV) and betanodavirus. The antigens from these viruses may be proteins or immunologically effective fragments thereof. Particularly preferred are surface proteins or immunologically effective fragments of these surface proteins.

In certain embodiments, the antigen is a capsid protein of a non-enveloped virus, such as, for example PMCV. Thus, in some embodiments, the antigen is a Piscine Myocarditis Virus (PMCV) ORF-1 antigen, such as a protein comprising SEQ ID NO: 1 or a sequence that it at least 90% identical to SEQ ID NO: 1, e.g., at least 95% identical, at least 96% identical, at least 97% identical, at least 98% identical or at least 99% identical to SEQ ID NO: 1. The sequence may differ from SEQ ID NO: 1 by deletions, insertions, or substitutions. Preferably, at least some substitutions are conservative substitutions. In certain embodiments, at least 50% (i.e., at least 60%, or at least 70%, or at least 80%, or at least 90%, or at least 95%, or at least 99%, or 100%) of the substituted amino acids are conservative substitutions.

In other embodiments, the antigen comprises a protein comprising, from N- to C-terminus:
a. a sequence that is at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID NO: 34 or SEQ ID NO: 35, and
b. an amino acid sequence that is at least 95%, 96%, 97%, 98%, 99%, or 100% identical to any one of SEQ ID NOs: 25-33, wherein,
i) said protein lacks SEQ ID NO: 15 or a sequence at least 90% identical to SEQ ID NO: 15; and/or
ii) compared to SEQ ID NO: 1, said protein comprises an internal deletion that is at least four consecutive amino acids long. In certain embodiments, the protein is at least 90% identical to SEQ ID NO: 11 or SEQ ID NO: 12.

In the embodiments where the Piscine Myocarditis Virus (PMCV) ORF-1 antigen lacks SEQ ID NO: 15, it should be understood that a larger deletion, e.g., a C-terminal truncation can also be made in the antigen. In certain embodiments, such larger C-terminal truncation comprises, or consists of, SEQ ID NO: 13.

In certain embodiments, the protein that is at least 95% identical to SEQ ID NO: 25 and lacks SEQ ID NO: 14 or a sequence that is at least 90% identical to SEQ ID NO: 14. Preferably, the protein lacks one or more of SEQ ID NO: 4, SEQ ID NO: 5, SEQ ID NO: 15 or a sequence that is at least 90% identical thereto.

In certain other embodiments, the protein of the invention includes the sequence that is at least 95% identical to SEQ ID NO: 26 and lacks SEQ ID NO: 14 or a sequence that is at least 90% identical to SEQ ID NO: 14. The protein may include the sequence that is at least 95% identical to SEQ ID NO: 26 and lacks SEQ ID NO: 14 or a sequence that is at least 90% identical to SEQ ID NO: 14 and further lack one or more of SEQ ID NO: 5, 15, or 24 or a sequence that it at least 90% identical thereto.

In other embodiments, the protein includes the sequence that is at least 95% identical to SEQ ID NO: 27, and lacks SEQ ID NO: 14 or a sequence that is at least 90% identical to SEQ ID NO: 14. The protein may include the sequence that is at least 95% identical to SEQ ID NO: 27 and lacks SEQ ID NO: 14 or a sequence that is at least 90% identical to SEQ ID NO: 14 and further lack one or more of SEQ ID NO: 3, 15, or 24 or a sequence that it at least 90% identical thereto.

In other embodiments, the protein includes the sequence that is at least 90% identical to SEQ ID NO: 28. Optionally, the protein lacks one or more of SEQ ID NOs 3, 4, or 24 or a sequence that it at least 90% identical thereto.

In other embodiments, the protein comprises SEQ ID NO: 29 or SEQ ID NO: 30 or a sequence at least 95%, or 96%, or 97% or 98% or 99% identical to any one of these sequences. Preferably, the protein lacks at least one of SEQ ID NO: 3 or SEQ ID NO: 24, or an amino acid sequence that is at least 90% identical to SEQ ID NO: 3 or SEQ ID NO: 24.

In other embodiments, the protein comprises SEQ ID NO: 32 or a sequence at least 95%, or 96%, or 97% or 98% or 99% identical thereto and, optionally, lacks at least one of SEQ ID NO: 14 or SEQ ID NO: 15, or an amino acid sequence that is at least 90% identical to SEQ ID NO: 14 or SEQ ID NO: 15.

In other embodiments, the protein comprises SEQ ID NO: 33 or a sequence at least 95%, or 96%, or 97% or 98% or 99% identical thereto and optionally, lacks SEQ ID NO: 24 an amino acid sequence that is at least 90% identical to SEQ ID NO: 24.

In any of the embodiments described above, the protein may further optionally lack at least one of SEQ ID NOs 2 and 13, or an amino acid sequence that is at least 90% identical to SEQ ID NO: 2 or 13.

In other embodiments, the protein includes SEQ ID NO: 15 or a sequence at least 90% identical thereto and anyone of SEQ ID NOs 4, 5, 25, 26 27, 29, 30, 32, (or an amino acid sequence that is at least 90% identical to SEQ ID NO: 4, 5, 25, 26 27, 29, 30, or 32) wherein, compared to SEQ ID NO: 1, said protein comprises an internal deletion that is at least four consecutive amino acids long, and, optionally, wherein said protein comprises a linker in place of said internal deletion.

Compared to SEQ ID NO: 1, said Piscine Myocarditis Virus (PMCV) ORF-1 antigen may comprise an internal deletion that is at least four consecutive amino acids long. In general, the internal deletion may be up to 250 amino acids long. Preferably, the internal deletion is about 10 amino acids long or 10 to 250 amino acids long, or 25 to 250 amino acids long, or 50 to 250 amino acids long, or 100 to 250 amino acids long, or 150-250 amino acids long, or 10 to 200 amino acids long, or 25 to 200 amino acids long, or 50 to 200 amino acids long, or 100 to 200 amino acids long, or 150 to 200 amino acids long or about 200 amino acids long, or about 50 amino acids long or 50 to 150 amino acids long, or 50-100 amino acids long or about 100 amino acids long. In certain embodiments, the internal deletion comprises or consists of SEQ ID NO: 2, SEQ ID NO: 3 or SEQ ID NO: 14.

In certain embodiments, the linker may be present in place of said internal deletion. The lengths of the internal deletion and the linker do not need to be the same. For example, the linker may be 3-50 amino acids long, or 3-40 amino acids long, or 3-30 amino acids long, or 3-20 amino acids long, or 3-10 amino acids long, or about 5 amino acids long, or about 10 amino acids long, or about 20 amino acids long. The exact sequence of the linker is not very important. In general, preferable amino acids are polar uncharged or charged residues, which constitute approximately 50% of naturally encoded amino acids. More specifically, Threonine, Serine, and Glycine may provide good flexibility due to their small sizes, and also help maintain stability of the linker structure in the aqueous solvent through formation of hydrogen bonds with water. In certain embodiments the linker is glycine and/or serine rich. In certain embodiments, the linker is 5 amino acids long and may comprise SEQ ID NO: 36. SEQ ID NO: 36 may be encoded by SEQ ID NO: 37.

In yet other embodiments, the protein lacks SEQ ID NO: 13 or a sequence that is at least 90% identical to SEQ ID NO: 13 and contains SEQ ID NO: 25 or a sequence at least 95%, or 96%, or 97% or 98% or 99% identical to SEQ ID NO: 25.

In certain embodiments, the protein comprises SEQ ID NO: 11, 12 or 46 or a sequence that is at least 90%, or at least 91%, or at least 92%, or at least 93%, or at least 94%, or at least 95%, or at least 96%, or at least 97%, or at least 98%, or at least 99%, or 100% identical to SEQ ID NO: 11 or SEQ ID NO: 12 or SEQ ID NO: 46.

In certain preferred embodiments, the fusion protein comprises SEQ ID NO: 9, 10, or 47 or a sequence that is at least 90%, or at least 91%, or at least 92%, or at least 93%, or at least 94%, or at least 95%, or at least 96%, or at least 97%, or at least 98%, or at least 99%, or 100% identical to SEQ ID NO: 9, 10, or 47. Preferably, the differing amino acids are substitutions and at least half of these substitutions (e.g., at least 60%, at least 70%, at least 80% at least 90%, at least 95%, at least 98%, at least 99% or 100%) are conservative substitutions.

In the second aspect, the disclosure provides a nucleic acid sequence that encodes the protein according to any of the embodiments of the first aspect or the fusion protein according to any of the embodiments of the second aspect of the invention. The nucleic acid sequence according to the third aspect of the invention may, in some embodiments, comprise SEQ ID NO: 16 or is at least 90% (at least 91%, or at least 92%, or at least 93%, or at least 94%, or at least 95%, or at least 96%, or at least 97%, or at least 98%, or at least 99%) identical thereto.

In the third aspect the invention provides an expression cassette comprising the nucleic acid sequence according to any embodiment of the second aspect of the invention. Preferably, the nucleic acid sequence according to any embodiment of the second aspect of the invention is under operable control of a first promoter. The first promoter may be selected from such exemplary promoters as simian virus 40 early promoter (SV40), cytomegalovirus immediate-early promoter (CMV), human Ubiquitin C promoter (UBC), human elongation factor 1a promoter (EF1A), mouse phosphoglycerate kinase 1 promoter (PGK), and chicken β-Actin promoter coupled with CMV early enhancer (CAGG). Generally, the expression cassette of the invention also comprises a polyadenylation signal that terminates transcription of the nucleic acid sequence encoding the antigen.

Optionally, the cassette may also comprise a nucleic acid sequence encoding a molecular immunomodulator under operative control of a second promoter. Generally, the second promoter should be able to initiate transcription in the host organism. In the embodiments where the host is a salmonid, such as Salmo salar, suitable promoters include, without limitations, simian virus 40 early promoter (SV40), cytomegalovirus immediate-early promoter (CMV), human Ubiquitin C promoter (UBC), human elongation factor 1a promoter (EF1A), mouse phosphoglycerate kinase 1 promoter (PGK), and chicken β-Actin promoter coupled with CMV early enhancer (CAGG). Generally, the expression cassette of the invention also comprises a polyadenylation signal that terminates transcription of the nucleic acid sequence of the invention.

In a fourth aspect, the application discloses a vector comprising the cassette according to any of the embodiments of the third aspect of the invention. Different vectors suitable for the invention are known in the art, including both plasmid vectors and viral vectors. Suitable plasmids include, without limitations pUC-based vectors, pVAX-vectors, pcDNA-vectors, NTC-vectors. In a set of preferred embodiments, the vector is NTC9385R (Nature Technology Corporation) or a variant thereof, as described in the Examples.

In other embodiments, a relatively new “doggybone” or dbDNA™ plasmid may be used as a vector. dbDNA™ plasmids as well as the process of making these plasmids have been described at least in WO2018033730, WO2016034849, WO2019193361, WO2012017210 and WO2021161051. The advantage of this approach is that the vector can be synthesized in a cell-free process thus improving manufacturing efficiency. The cell-free process preferably involves amplification of the template via strand displacement replication. This synthesis releases a single stranded DNA, which may in turn be copied into double stranded-DNA, using a polymerase. Alternatively, strand displacement can be achieved by supplying a DNA polymerase and a separate helicase. Replicative helicases may open the duplex DNA and facilitate the advancement of the leading-strand polymerase. The resulting double-stranded DNA concatemer is enzymatically cut and ligated thus forming the doggybone-like shape DNA construct.

Suitable viral vectors include, without limitations, alphaviruses such as SAV, rhabdoviruses such as VHSV and IHNV, paramyxoviruses such as ASPV, adenoviruses, poxviruses such as Salmon gill poxvirus, and the like. These viruses can be genetically modified to remove the parts of the viral genomes responsible for replication. Thus, the resulting viruses would be infectious to fish cells and suitable for production of the antigen, but not be pathogenic.

In the embodiments where the cassette does not contain the nucleic acid sequence encoding the molecular immunomodulator, such nucleic acid sequence may still be present in the vector.

In certain embodiments, suitable for both the vector of this aspect and the cassette of the third aspect, the molecular immunomodulator is interferon, preferably IFNb or the like and comprises SEQ ID NO: 6 (Salmo salar Interferon B, or IFN-b protein) or SEQ ID NO: 50 (Salmo salar Interferon B1, or IFNb1 protein) or a sequence that is at least 95% (e.g., at least 96% or at least 97% or at least 98% or at least 99%) identical to SEQ ID NOs 6 or 50, wherein said interferon B is encoded by a nucleic acid sequence that is 65-90% identical to SEQ ID NO: 18 or wherein said interferon B1 is encoded by a nucleic acid sequence that is 65-90% identical to SEQ ID NO: 19, and wherein codon frequency is preserved in said nucleic acid sequence.

In certain embodiments, the nucleic acid sequences encoding the interferon are 70-79% or 73-77% identical to SEQ ID NO: 18 or SEQ ID NO: 19, with a proviso that codon frequency is preserved in said nucleic acid sequences encoding the interferon.

As disclosed above, the frequencies of at least 50% of codons in the nucleic acid sequence encoding the interferon do not deviate by more than 40% from the frequencies of the same codons in the genome of the host. In certain embodiments, the frequencies of at least 40% codons in the nucleic acid sequence encoding the interferon do not deviate by more than 30% from the frequencies of the same codons in the genome of the host, and/or the frequencies of at least 30% codons in the nucleic acid sequence encoding the interferon do not deviate by more than 25% from the frequencies of the same codons in the genome of the host, and/or the frequencies of at least 25% codons in the nucleic acid sequence encoding the interferon do not deviate by more than 20% from the frequencies of the same codons in the genome of the host. If the host is Salmo salar, codon frequencies in Salmo salar are provided in Table 1.

In certain embodiments, the nucleic acid sequences comprise SEQ ID NO: 17 or SEQ ID NO: 20 or are at least 90% identical (i.e., over 91% identical, over 92% identical, over 93% identical, over 94% identical, over 95% identical, over 96% identical, over 97% identical, over 98% identical, or over 99% identical) to one of SEQ ID NO: 17 or SEQ ID NO: 20 and said nucleic acid sequences encode SEQ ID NO: 6 or 50 or an amino acid sequence at least 95% identical thereto, as described above.

Accordingly, in certain embodiments, the vector comprises both the fusion protein and the molecular adjuvant, wherein:
a. the fusion protein comprises SEQ ID NO: 9, 10, or 47 or a sequence that is at least 90%, or at least 91%, or at least 92%, or at least 93%, or at least 94%, or at least 95%, or at least 96%, or at least 97%, or at least 98%, or at least 99%, or 100% identical to SEQ ID NO: 9, SEQ ID NO: 10 or SEQ ID NO: 47. Preferably, the differing amino acids are substitutions and at least half of these substitutions (e.g., at least 60%, at least 70%, at least 80% at least 90%, at least 95%, at least 98%, at least 99% or 100%) are conservative substitutions; and
b. SEQ ID NO: 17 or SEQ ID NO: 20; and, optionally
c. the vector is a plasmid vector, preferably NTC9385R or a variant thereof.

In certain embodiments, the vector may contain the expression cassette as described above and an additional antigen, e.g. a salmonid alphavirus antigen. It is also possible that the vector would contain an additional nucleic acid sequence encoding a different molecular immunomodulator.

The vector according to any embodiment of this aspect of the invention may optionally include amino acid sequences encoding additional antigen(s). In general, these additional antigens may have viral, bacterial, protozoal, or parasitic origin. Suitable viruses include PMCV, SAV, ISA, IPNV, ASPV (Atlantic salmon poxvirus), IHNV (Infectious hematopoietic necrosis virus), VHSV (Viral hemorrhagic septicemia virus) and PRV. Antigens from enveloped and non-enveloped viruses may be included. In certain embodiments, the antigen is a viral structural protein or a capsid protein, or an outer surface protein. Fragments of these proteins capable of eliciting protective immune response are also suitable.

Suitable bacteria include, without limitations, Aeromonas salmonicida subs. Salmonicida, Vibrio (Listonella) anguillarum, Vibrio salmonicida, Moritella viscosa, and Yersinia ruckeri. Again, proteins present on the outer surface of the bacteria are preferred antigens, as well as these proteins capable of eliciting protective immune response.

Suitable parasites include sea lice (family Caligidae, preferably genera Lepeophtheirus or Caligus). Proteins originating from sea lice and capable of eliciting protective immune response have been described and include gut peptides or fragments thereof including without limitations SEQ ID NO: 21 or a sequence at least 90% identical thereto.

One of ordinary skill in the art would appreciate that the differences between the described amino acid sequences and the reference amino acid sequences (whether in the context of the fusion protein according to any embodiment of the first aspect, or the molecular immunomodulator as suitable in certain embodiments of the third or fourth aspect of the invention) may be in the form of insertions, deletions, or substitutions. Preferably, the mutations are substitutions, and more preferably, at least some of these substitutions are conservative substitutions.

The skilled person will further acknowledge that alterations of the nucleic acid sequence resulting in modifications of the amino acid sequence of the protein it codes may have little, if any, effect on the resulting three-dimensional structure of the protein. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in the substitution of one negatively charged residue for another, such as aspartic acid for glutamic acid, or one positively charged residue for another, such as lysine for arginine, can also be expected to produce a protein with substantially the same functional activity.

The following six groups each contain amino acids that are typical conservative substitutions for one another: [1] Alanine (A), Serine (S), Threonine (T); [2] Aspartic acid (D), Glutamic acid (E); [3] Asparagine (N), Glutamine (Q); [4] Arginine (R), Lysine (K), Histidine (H); [5] Isoleucine (1), Leucine (L), Methionine (M), Valine (V); and [6] Phenylalanine (F), Tyrosine (Y), Tryptophan (W), (see, e.g., US Patent Publication 20100291549).

In certain embodiments, at least 50%, or at least 60%, or at least 70% or at least 80% or at least 90% or at least 95% or all 100% of amino acids differing from the reference sequence are conservative substitutions.

Protein and/or nucleic acid sequence identities according to any of the embodiments described herein can be evaluated using any of the variety of sequence comparison algorithms and programs known in the art. For sequence comparison, typically one sequence acts as a reference sequence (e.g., a sequence disclosed herein), to which test sequences are compared. A sequence comparison algorithm then calculates the percent sequence identities for the test sequences relative to the reference sequence, based on the program parameters.

The percent identity of two amino acid or two nucleic acid sequences can be determined for example by comparing sequence information using the computer program GAP, i.e., Genetics Computer Group (GCG; Madison, WI) Wisconsin package version 10.0 program, GAP (Devereux et al. (1984), Nucleic Acids Res. 12: 387-95). In calculating percent identity, the sequences being compared are typically aligned in a way that gives the largest match between the sequences. The preferred default parameters for the GAP program include: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14: 6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps.

Sequence identity and/or similarity can also be determined by using the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (BESTFIT, FASTA, and TFASTA in the Wisconsin Genetics Software Package, Genetics Computer Group, 575 Science Drive, Madison, Wis.).

Another example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CAB/OS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program obtained from Altschul et al., 1996, Methods in Enzymology 266:460-480. WU-BLAST-2 uses several search parameters, most of which are set to the default values.

The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xᵤ set to 16, and X_{g} set to 40 for database search stage and to 67 for the output stage of the algorithms. Gapped alignments are triggered by a score corresponding to about 22 bits.

A person of ordinary skill in the art will appreciate that multiple methods exist for making the amino acid sequences according to the first aspect of the invention, the nucleic acids according to the second aspect of the invention, the expression cassettes of the third aspect of the invention and the vectors of the fourth aspect of the invention. For example, the nucleic acid sequences may be designed using software tools, e.g., CLC Main Workbench, and synthesized artificially or generated using targeted mutagenesis. These sequences may be subcloned into expression cassettes and vectors using genetic engineering techniques widely available to one skilled in the art. See, e.g., Molecular cloning: a laboratory manual (Sambrook & Russell: 2000, Cold Spring Harbor Laboratory Press; ISBN: 0879695773), and: Current protocols in molecular biology (Ausubel et al., 1988+ updates, Greene Publishing Assoc., New York; ISBN: 0471625949).

In the fifth aspect, the application discloses a vaccine comprising the vector according to any of the embodiments of the fourth aspect of the invention. The vaccine may be monovalent or multivalent. Generally, one dose of the monovalent vaccine may contain at least 1 μg of the vector according to any of the embodiments of the fourth aspect. In certain embodiments, one dose of the vaccine may contain between 1 μg and about 25 μg of the vector. In other embodiments, one dose of the vaccine may contain between 1 and about 20 μg of the vector, or between about 5 and about 20 μg of the vector or between about 5 and about 15 μg of the vector or between about 10 and about 20 μg of the vector or between about 5 and about 10 μg of the vector.

Multiple antigens suitable for the vaccine according to the invention have been described above. These antigens may be administered in the form of DNA vaccines, including vectors and expression cassettes described above, in the third and fourth aspects of the invention. In other embodiments, these antigens may be administered in forms of subunits (i.e., purified or partially purified proteins). In yet other embodiments, the antigens may comprise inactivated or attenuated organisms.

In addition to the antigens, the vaccine of the invention may further comprise adjuvants, i.e., the substances that enhance or modulate immune response, in addition to the molecular immunomodulator encoded by SEQ ID NO: 6 or an amino acid sequence that is 95% identical thereto.

Suitable adjuvants are well known in the art. Suitable non-limiting adjuvants include saponins (e.g., Quil A), alum, CpG oligonucleotides, poly I:C, oligoribonucleotides, cytokines, glycolipids such as BAY©1005, quaternary amines such as dimethyl dioctadecyl ammonium bromide (hereinafter, “DDA”). Complexes comprising the saponin, the sterol (e.g., cholesterol), and, optionally, a phospholipid, have been described in the art. Combinations of CpG oligonucleotides and Saponin, CpG and cholesterol, and CpG and alum have been reported to elicit synergistic effects.

In addition to the antigens and optional additional adjuvants, the vaccine may also comprise a suitable pharmaceutical carrier. The appropriate carrier is evident to those skilled in the art and will depend in large part upon the route of administration. Additional components that may be present in this invention are adjuvants, preservatives, surface active agents, chemical stabilizers, suspending or dispersing agents. Typically, stabilizers, adjuvants and preservatives are optimized to determine the best formulation for efficacy in the target subject.

In certain embodiments, the vaccine may include liposomal adjuvant and/or carrier to facilitate the transport of the vector across the cell membrane and thus result in an increased expression of the antigen and/or the molecular immunomodulator. A suitable non-limiting example of such liposomal adjuvant/carrier system is described, for example, in the U.S. Pat. No. 10,456,459.

In a sixth aspect, the application discloses a method of protecting a salmonid against an infection, the method comprising administering to the salmonid in need thereof the vaccine according to any of the embodiments of the fifth aspect of the invention. Thus, the disclosure also provides the vaccine according to any of the embodiments of the fifth aspect for use in protecting a salmonid against an infection.

It should be clear to one of ordinary skill in the art that the infection that the antigen(s) present in the vaccine dictate what invention the vaccine would protect against. Thus, for example and without limitation, if the vaccine comprises a nucleic acid sequence encoding a PMCV antigen, then the vaccine would be used against infection caused by PMCV.

The vaccine according to the invention may be administered to the salmonid by a variety of routes, including, without limitation, intramuscularly. Preferably, the vaccine is administered in a microdose such that the volume of one dose is under 500 μl, or under 400 μl, or under 300 μl or under 200 μl or about 100 μl or under 100 μl, or about 50 μl or about 25 μl.

The vaccine disclosed herein may be used in protecting multiple salmonid species against an infection. Suitable salmonids include, without limitations, Atlantic salmon (Salmo salar), coho salmon (Oncorhynchus kisutch), rainbow trout (Oncorhynchus mykiss), sockeye salmon (Oncorhynchus nerka), Chinook salmon (Oncorhynchus tshawytscha) and other species.

Salmonids of different ages (or weights) may be vaccinated according to the invention. In certain embodiments, the salmonid weighs between about 15 and about 200 grams at the time of vaccination. Thus, the weight of the salmonid at the time of the vaccination may be between about 25 and about 150 grams or between about 40 and about 110 grams or between about 50 and about 100 grams.

### EXAMPLES

### Example 1. Preparation of Surface-Targeting Expression System

All successful DNA vaccines for Atlantic salmon have been derived from enveloped viruses and are based on membrane-bound antigens. Exposure of the antigen on the surface of cells could therefore be important for efficacy.

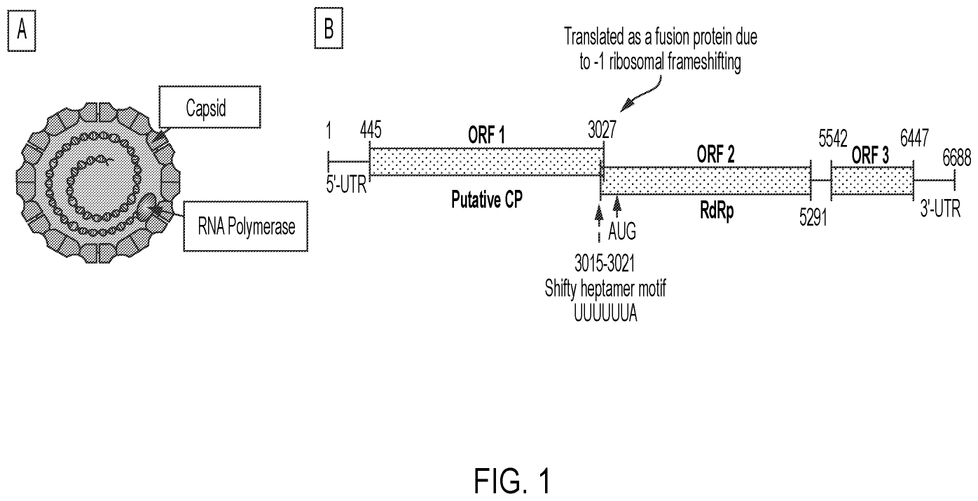

PMCV is a naked virus, containing a dsRNA genome of 6.7 kb with three predicted open reading frames (FIG. 1). The capsid protein (CP) is translated from ORF1, while ORF2 encodes the RNA dependent RNA polymerase (RdRp). The function of the ORF3 protein is still unknown, but it encodes a protein with cytotoxic effect when over-expressed in cell culture and is not necessary for assembly of viral particles.

DNA vaccines expressing the PMCV ORF1 (capsid protein) and PMCV ORF3 (protein of unknown function) had already been tested without providing protection (data not shown). Since PMCV is a naked virus with no transmembrane regions predicted for the PMCV ORF1 protein, and it was demonstrated that the PMCV ORF1 protein would only be intracellularly expressed (data not shown), it was hypothesized that response may be elicited if the expressed ORF1 capsid protein is targeted to the cell membrane.

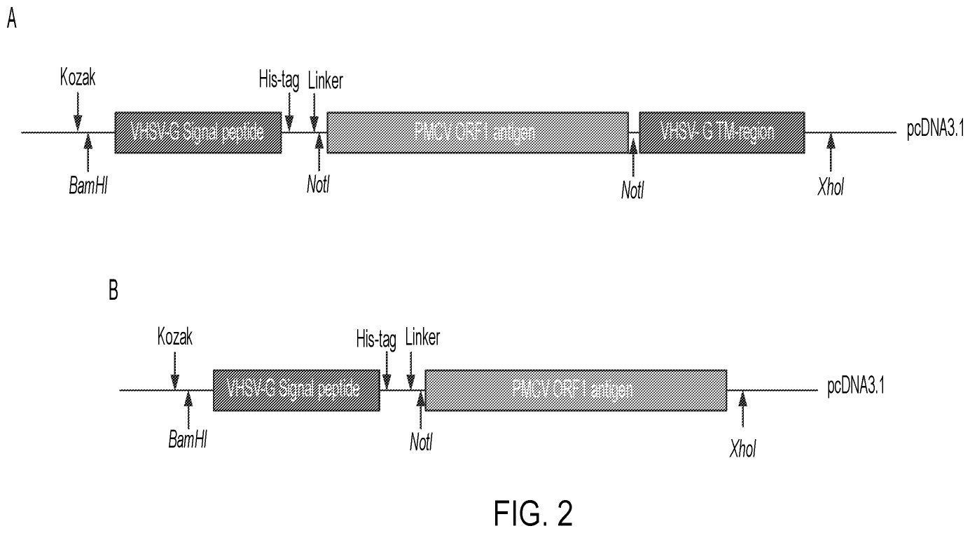

The aim of this experiment was to construct a DNA vaccine expressing the PMCV capsid protein (or parts of it) as a cell surface expressed antigen. The strategy was to fuse the N-terminal secretion signal peptide of the viral hemorrhagic septicemia virus G-protein (VHSV-G) according to SEQ ID NO: 7 upstream of the PMCV ORF1 antigen N-terminus. Additionally, the C-terminal transmembrane domain of VHSV-G according to SEQ ID NO: 8 was fused downstream of the antigen C-terminus, to incorporate the antigen at the cell surface (von Gersdorff Jorgensen et al., 2012), as illustrated in FIG. 2A. A secreted version of the vaccine antigen was also prepared by only using N-terminal secretion signal peptide and omitting the use of the C-terminal transmembrane domain (FIG. 2B).

Additional constructs were prepared similarly to the construct illustrated in FIG. 2A, except where instead the signal peptide sequence and transmembrane domain sequence of the Atlantic salmon paramyxovirus fusion protein (SEQ ID NOs: 38 and 39, respectively) or Hemagglutinin (HE) protein of Infectious salmon anemia virus (ISAV) (SEQ ID NOs: 40 and 41, respectively) were used.

Routing of antigen expression was studied by immunofluorescence (IF) staining 6 days after transfection of CHH-1 cells with various DNA vaccine constructs. Transfection was done using the LIPOFECTAMIN® 3000 kit (Thermo Fisher) in 12-well plates. The pVAX1 expression vector (Invitrogen) was utilized to construct the DNA vaccines in this experiment, and 1 μg of plasmid DNA was used for transfection. Cells were fixed either with 3.7% formaldehyde only for surface antigen-expression or fixed with 3.7% formaldehyde followed by permeabilization of cells using 0.1% Triton X-100 for staining of intracellularly expressed proteins. Vaccine antigen was detected both using a monoclonal antibody and polyclonal antibodies against PMCV ORF1. Appropriate fluorescent-labeled secondary antibodies (Rabbit anti-mouse FITC (DAKO F0261) or Polyclonal goat anti-rabbit Alexa Fluor Plus 488 (Invitrogen A32731) were used for detection. Staining permeabilized cells detecting intracellularly expressed antigen was routinely included as a positive control of the protocol.

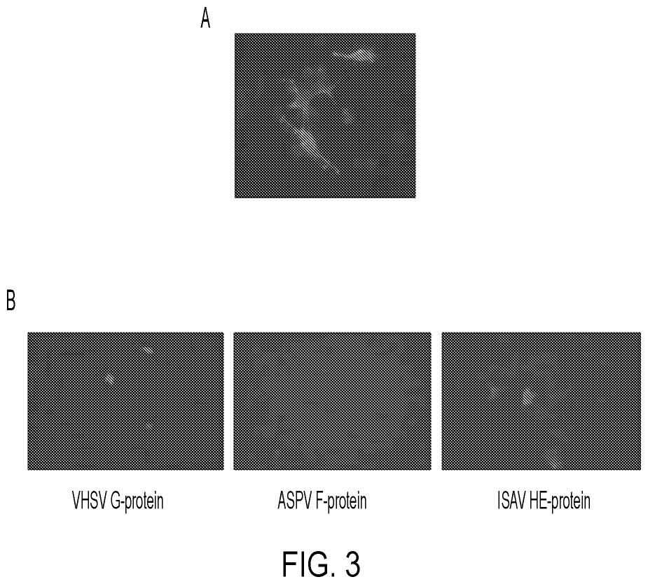

As shown in FIG. 3, the use of VHSV G-protein could be used for routing expression of PMCV ORF-1 antigen to the cell surface. Using signal protein and transmembrane domain from Hemagglutinin (HE) protein of Infectious salmon anemia virus also resulted in membrane staining of PMCV ORF1 protein, while using regions from the fusion protein of Atlantic Salmon Paramyxovirus was not successful.

In the next experiment, in vivo testing in Atlantic salmon (Salmo salar) was conducted. Two variants of the antigen were tested, the full-length capsid protein (G-ORF1) and a truncated capsid protein (G-ORF1-DeltaD) expressing the first half of the capsid protein (PMCV ORF1 position 1-1293 of SEQ ID NO: 16). Both antigens were expressed both in a standard expression vector (pcDNA3.1) and as an alphavirus replicon vaccine based on salmonid alphavirus 3 (SAV-3). PBS and pcDNA-eGFP were included as negative vaccine control groups.

For all vaccines in the present study the antigen encoding genes were inserted downstream of a CMV promoter in the eukaryotic expression vector pcDNA3.1(+) (Invitrogen). All plasmids were diluted in sterile phosphate-buffered saline (PBS) to 10 μg per 50 μl injection volume. All fish received one intramuscular injection on each side of the fish and a total dose of 20 μg. Two non-immunized control groups were included, one group received 20 μg of a control vaccine (eGFP in pcDNA3.1) and one group received 2×50 μl PBS.

TABLE 2

| | | | | Final conc | |
|---|---|---|---|---|---|
| Vaccine | | Detailed description | Plasmid | construct | Dose μg |
| group | Description | plasmid encoding antigen | backbone | (μg/ml) | (2 × 0.05 ml) |
| 1 | G-ORF1-Delta D | G-PMCV_ORF1_1-1293 | pcDNA3.1(+) | 200 | 20 |
| | | (Delta D) | | | |
| 2 | G-ORF1 | G-PMCV_ORF1_full_ | pcDNA3.1(+) | 200 | 20 |
| | | 1-2583 | | | |
| 3 | repSAV-G- | G-PMCV_ORF1_1-1293 | pcDNA3.1(+) | 200 | 20 |
| | ORF1-Delta D | (Delta D) in SAV replicon | | | |
| 4 | repSAV-G-ORF1 | G-PMCV_ORF1_full_ | pcDNA3.1(+) | 200 | 20 |
| | | 1-2583_in SAV replicon | | | |
| 5 | eGFP | enhanced Green | pcDNA3.1(+) | 200 | 20 |
| | | Fluorescent Protein | | | |
| | | (control) | | | |
| 6 | PBS | | | | NA |

The fish (n=45 per group) were kept in freshwater and had an average weight of 48 grams. Prior to vaccination, the fish were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 49 days at 12° C. (light:dark 12:12) before the fish were anaesthetized again using Tricain (PHARMAQ) and challenged with infectious PMCV by intraperitoneal injection of 0.1 ml of homogenized spleen (isolate ID AL V1223 Spleen) originating from a Norwegian field outbreak of CMS. The fish were then sampled at three timepoints; 10 days, 20 days and 50 days post challenge (n=15 per group, per sampling-point). Heart ventricle and kidney were collected on RNALATER© from all sampled fish for subsequent RNA extraction. Hearts (ventricle and atrium) were also fixed in formalin from all fish on the last sampling point (day 50) for histopathological analysis. RNA was extracted from all samples stored on RNALATER® and analyzed by Real-Time RT-PCR for presence of PMCV RNA using a commercially available service from PHARMAQ Analytiq (Bergen, Norway). Hearts stored on formalin were sectioned and histopathology was scored from 0 to 3 according to severity, where 0 indicates no pathologic finding and 3 indicate presence of severe pathology (service provided by PHARMAQ Analytiq, Bergen, Norway).

Vaccines expressing the full-length ORF1 of PMCV routed to the cell surface demonstrated significant protection against challenge compared to control groups (One-way ANOVA, with Dunnett's post test for histology in atrium, p<0.0001). This was shown both as increased Ct values in kidney and heart early after infection (results not shown), and reduced histopathology score 50 days after infection (Table 3). Antigen based on the first half of the capsid protein (G-ORF1-Delta D; PMCV ORF1 position 1-1293) failed to provide protection, and no additional increase in protection was provided using the salmon alphavirus replicon in contrast to the standard expression vector.

TABLE 3
Histopathological score in the heart atrium at 50 days post
challenge with PMCV.

| | | Mean | Standard | | p-value |
|---|---|---|---|---|---|
| Vaccine- | | histoscore, | deviation, | | (compared |
| group | Description | Atrium | histoscore | N | to PBS) |
| 6 | PBS | 2.29 | 0.78 | 15 | NA |
| 5 | eGFP | 2.21 | 0.67 | 15 | 0.99 |
| 4 | repSAV-G-ORF1 | 1.49 | 0.88 | 15 | 0.028 |
| 3 | repSAV-G-ORF1- | 2.31 | 0.69 | 15 | 0.99 |
| | Delta D | | | | |
| 2 | G-ORF1 | 1.11 | 0.83 | 15 | 0.0005 |
| 1 | G-ORF1-Delta D | 2.35 | 0.83 | 15 | 0.99 |

Histopathological score in the heart atrium at 50 days post challenge with PMCV. The indicated p-value was calculated by comparing individual groups with the PBS control group using a One-way ANOVA, with Dunnett's post test.
NA = Not applicable.

The DNA vaccine candidate targeting the full-length antigen to the cell-surface gave an approximately 50% reduction of histopathological score and was accompanied by a reduced viral load (qPCR) in kidney and heart.

A follow up in vivo trial compared the efficacy of DNA vaccines expressing the PMCV ORF1 full-length protein as a cell surface expressed antigen, secreted antigen or an intracellularly expressed antigen. In addition, a codon-optimized version of the full-length capsid protein encoded by SEQ ID NO: 45 was included. The study contained two parallel immunization tanks with five groups each of Atlantic salmon (n=30 per group, per tank).

For all vaccines in the present study the antigen encoding genes were inserted downstream of a CMV promoter in the eukaryotic expression vector NTC9385R (Nature Technology Corporation). All plasmids were diluted in sterile phosphate-buffered saline (PBS) to 10 μg per 50 μl injection volume. All fish received one intramuscular injection on each side of the fish and a total dose of 20 μg. A non-immunized control group was included and received 2×50 μl PBS, as summarized in Table 4.

TABLE 4

| | | | | | Final | |
|---|---|---|---|---|---|---|
| | | | | Antigen | conc | Dose μg |
| Vaccine | | Description plasmid | Plasmid | expressed | construct | (2 × 0.05 |
| group | Description | encoding antigen | backbone | at | (μg/ml) | ml) |
| 1 | G-ORF1 | G-PMCV_ORF1_full_1- | NTC9385R | Cell-surface | 200 | 20 |
| | | 2583 | | | | |
| 2 | ORF1 | PMCV_ORF1_full_1- | NTC9385R | Intracellular | 200 | 20 |
| | | 2583 | | | | |
| 3 | G-ORF1 | G-PMCV_ORF1_full_1- | NTC9385R | Cell-surface | 200 | 20 |
| | Codon opt | 2583-Codon optimized | | | | |
| 4 | G-ORF1 w/o | G-PMCV_ORF1_full_1- | NTC9385R | Secreted | 200 | 20 |
| | TM | 2583 without TM | | | | |
| | | domain | | | | |
| 5 | PBS | | NA | NA | | |

The indicated p-value was calculated by comparing individual groups with the PBS control group using a One-way ANOVA, with Dunnett's post test.
NA = Not applicable.

The fish were kept in freshwater and had an average weight of 26 grams at the time of vaccination. During administration of the test vaccines, the fish were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 39 days at 12° C. (light:dark 12:12) before the fish were anaesthetized again using Tricain (PHARMAQ) and challenged with infectious PMCV by intraperitoneal injection of 0.1 ml of homogenized heart (isolate ID AL V1273 Heart) originating from a Norwegian field outbreak of CMS. The fish (n=15 per group, per time-point) were then sampled 21 days and 56 days post challenge. Heart ventricle and kidney were collected on RNALATER© from all sampled fish for subsequent RNA extraction. Hearts (ventricle and atrium) were also fixed in formalin from all fish on the last sampling point (day 56) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 3 according to severity, where 0 indicates no pathologic finding and 3 indicate presence of severe pathology (service provided by PHARMAQ Analytiq, Norway).

The results showed that significant protection against PMCV infection was achieved with vaccines expressing the full-length ORF1 of PMCV routed to the cell surface (G-ORF1 and G-ORF1 codon optimized). Expression of PMCV ORF1 in its native form (intracellular protein) did not provide detectable protection. Expression by secretion provided some protection (no difference detected in kidney at day 20; See Tables 5, 6).

TABLE 5
Tank 1. Viral RNA in kidney 3 weeks post challenge.

| | | Mean | Standard | | p-value |
|---|---|---|---|---|---|
| Vaccine | | Ct-value, | deviation, | | (compared |
| group | Description | PMCV | Ct | N | to PBS) |
| 5 | PBS | 20.95 | 0.96 | 16 | NA |
| 1 | G-ORF1 | 23.02 | 2.17 | 15 | 0.0042 |
| 3 | G-ORF1 Codon opt | 23.09 | 1.67 | 15 | 0.0031 |
| 4 | G-ORF1 w/o TM | 20.74 | 2.02 | 15 | 0.99 |
| 2 | ORF1 | 19.92 | 1.41 | 15 | 0.30 |

The indicated p-value was calculated by comparing individual groups with the PBS control group using a One-way ANOVA, with Dunnett's post test.
NA = Not applicable.

TABLE 6
Tank 2. Viral RNA in kidney 3 weeks post challenge.

| | | Mean | Standard | | p-value |
|---|---|---|---|---|---|
| Vaccine | | Ct-value, | deviation, | | (compared |
| group | Description | PMCV | Ct | N | to PBS) |
| 5 | PBS | 20.05 | 0.73 | 15 | NA |
| 1 | G-ORF1 | 22.12 | 1.95 | 15 | 0.0005 |
| 3 | G-ORF1 Codon opt | 23.06 | 1.93 | 15 | <0.0001 |
| 4 | G-ORF1 w/o TM | 20.06 | 1.16 | 15 | >0.99 |
| 2 | ORF1 | 19.90 | 0.67 | 15 | >0.99 |

The indicated p-value was calculated by comparing individual groups with the PBS control group using a One-way ANOVA, with Dunnett's post test.
NA = Not applicable.

### Example 2: Preparation and Determination of Antigenic Activity of CMS Antigen

Expressing the PMCV ORF1 full-length protein on the cell membrane provided partial protection in the challenge model. Despite this finding, it was observed in vitro that expression levels of the antigen were markedly lower than expression levels of a control GFP-construct. The project has investigated whether expressing truncated versions of the capsid protein by deleting regions could increase expression levels and immunogenicity of the vaccine antigen.

Expression system described in Example 1 (using VHSV-G signal peptide and transmembrane domain sequences) was prepared with different constructs based on PMCV ORF-1 antigen.

The first studies expressing membrane-bound PMCV capsid antigen from the VHSV-G expression cassette showed that including the first part of the PMCV ORF1 protein sequence was pivotal for success. When the capsid protein was divided into thirds, membrane bound protein was successfully achieved only for constructs comprising the first third (data not shown).

Following construction of PMCV ORF1 capsid deletion variants, these were tested by IF-staining and Western blot of transfected cells. For all deletion constructs, the deleted PMCV ORF1 sequence was replaced with a GGGGS linker (SEQ ID NO: 36). The strategy for making the various deletion variants was as follows:
(1) In the first round, relatively large deletions were tested (IntDel 1-7; 200 amino acids in length, overlapping by 100 amino acids, except IntDel7 which was 162 amino acids long). As inclusion of some N-terminal sequence was found to be required for successful routing of expressed protein to cell membranes, the first deletion (IntDel 1) was made at nucleotide positions 298-897 within PMCV ORF1 (SEQ ID NO: 16).
(2) In the second round, smaller deletion-variants were made focusing on the region covered by IntDel 4-7 (IntDel 11-18, adjacent, non-overlapping 50 amino acids in length, starting within IntDel 4). The first of these deletions (IntDel 11) was constructed at nucleotide positions 1384-1533 within PMCV ORF1 (SEQ ID NO: 16).
(3) In the third round, antigen variants with very small adjacent overlapping deletions were made (IntDel 21-42; 10 amino acids in length, overlapping by 2 amino acids). The first of these deletions (IntDel 21) was constructed at nucleotide positions 1936-1965 within PMCV ORF1 (SEQ ID NO: 16).

Expression of PMCV ORF1 capsid deletion variants in CHH-1 cells by IF-staining and Western blot. The procedure for immunofluorescence staining of cells was as follows: CHH-1 cells were trypsinated and seeded into 12-well plates (150 000 cells per well and 1 ml cell-medium). The cells were incubated at 15° C. ON in a CO₂ incubator (5% CO₂). The cells were transfected the day after seeding using 1 μg plasmid/well and LIPOFECTAMIN® 3000 (Thermo Fisher Scientific) according manufacturer's instructions. The cells were incubated at 15° C. for 5 days in a CO₂ incubator (5% CO₂) before immunostaining of cells. The cell-medium was carefully removed from all wells, and all wells were washed carefully with PBS. Cells were fixed by incubation with 3.7% formaldehyde (diluted in PBS) for 15 min at RT. Cells were then blocked with 5% non-fat dry milk in PBS for 1 h. Both mouse anti-HIS (6×-His Tag Monoclonal Antibody (4A12E4) Fisher scientific; cat #37-2900 at 1:1500 dilution and mouse anti-PMCV ORF1 monoclonal antibody (mAb #10; 1:200 dilution) were used as primary antibodies and incubated at room temperature for 1 h. FITC-conjugated Polyclonal Rabbit Anti-mouse Immunoglobulin (DAKO F0261) was used as the secondary antibody at 1:1000 dilution and incubated for another 1 h. Staining of cells were evaluated by fluorescence microscopy.

Evaluating expression levels of PMCV ORF1 deletion constructs by Western blotting was done as follows: CHH-1 cells were trypsinated and seeded into 6-well plates (1 000 000 cells per well and 3 ml cell-medium). The cells were incubated at 15° C. ON in a CO₂ incubator (5% CO₂). The cells were transfected the day after seeding using 2.5 μg plasmid/well and LIPOFECTAMIN® 3000 (Thermo Fisher Scientific) according manufacturer's instructions. Cells were put back in sealed bags and incubated at 15° C. in a CO₂ incubator (5% CO₂) for six days before further analysis. Plates were then placed on ice and medium removed. Cells were first washed twice with ice-cold PBS. To prepare membrane lysates, cells were collected first adding 200 μl of 20 mM Tris-HCl, pH 7.5 with protease inhibitors, cells carefully scraped off and the cell suspensions transferred to pre-cooled tubes. The well was then flushed with another 100 μl to a total of 300 μl of cell suspension per transfection. As this is a detergent-free buffer, cells were lysed by passing cells through a syringe tip (25G, ˜5 times) and centrifuged at 12 000 rpm for 10 min at 4° C. The supernatants were discarded, and the membrane pellets dissolved in 300 μl RIPA lysis-buffer (Abcam, #156034) with 1× Protease inhibitor cocktail to release membrane-bound proteins. Samples were incubated for 30 min at 4° C. while re-suspending the pellet occasionally by flicking the tube. Tubes were centrifuged at 12 000 rpm for 10 min at 4° C. and the membrane-fraction lysates transferred to fresh tubes and kept on ice until immunoprecipitation. On ice, 1 μg of mouse IgG1anti 6×-His monoclonal antibody 4E3D10H2/E3 (Fisher Scientific #15442890, 1 mg/ml) was added to each sample and incubated for 4° C. overnight on a rotator. Protein G-coupled beads were prepared (25 μl/sample) by washing the immobilized beads 3-5× with ˜1 ml cold PBST and re-suspended in 25 μl of PBST with protease inhibitors per aliquote. On ice, 25 al of pre-equilibrated beads were added to each sample and the sample-beads mixture incubated for 1 hour at RT with rotation. The beads were washed with 500 μl of ice cold PBST with proteinase inhibitors 3-5 times to remove non-specific binding with gentle mixing of the beads between each wash. After the last wash, as much buffer as possible was removed from the beads and 40 μl of 1× Laemmli Sample Buffer (BioRad #161-0737) added (prepared by mixing 20 μl 2× Laemmli Sample Buffer with 2 μl β-mercaptoethanol and 18 μl dH2O). Samples were incubated at 70° C. for 10 minutes, quickly centrifuged and magnetized, and eluent moved to a new vial. Before loading on a 4-20% Criterion™ TGX Stain-Free™ Gel, all samples were denatured at 98° C. for 5 min. The gel was run for approximately 45 min at 200V and blotted using the TRANS-BLOT® TURBO™ Transfer System (BioRad), and TRANS-BLOT® TURBO™ Midi PVDF Transfer Packs #170-4157. 7 minutes turbo program was used. The membrane was immediately put in blocking-buffer (5% skimmed milk in PBST). Blocking was performed for 1 hour at room-temperature. The blot was incubated with primary antibody rabbit anti-delta PMCV ORF1 diluted 1: 500 in 1% skimmed milk/PBST over-night at 4° C. in rotator. The following day the blots were washed 2×15 minutes with PBST and incubated with secondary antibody polyclonal swine anti-rabbit (DAKO #P0217) immunoglobulin HRP diluted 1:1000 in 2% skimmed milk and PRECISION PROTEIN™ StrepTactin-HRP Conjugate 1:10000 (to visualize ladder) for 1 hour at RT. The blot was washed 2×15 minutes with PBST before detection of signal by Clarity western ECL substrate (BioRad #170-5060).

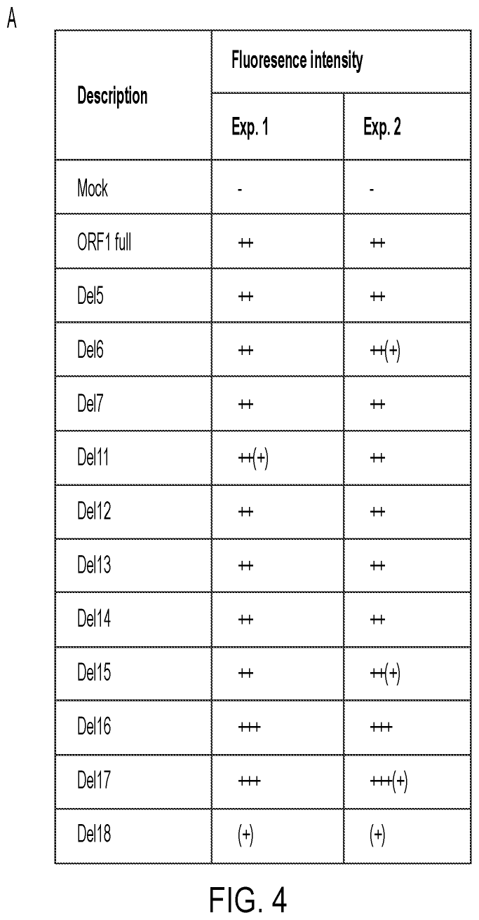

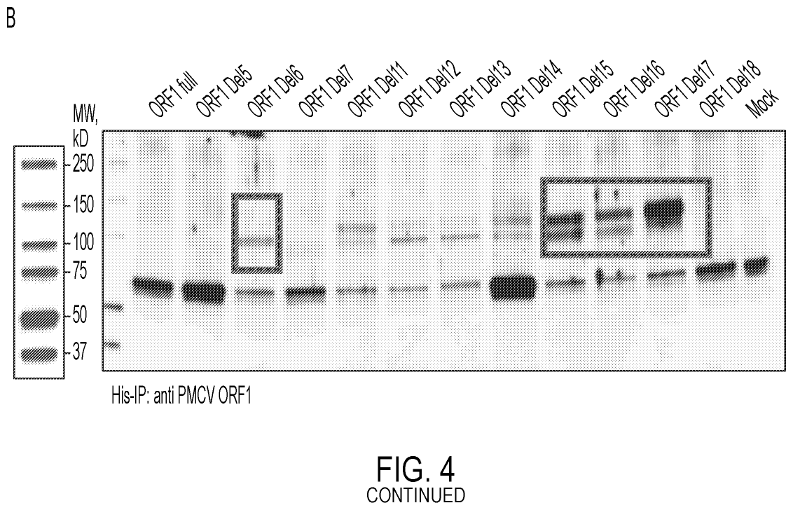

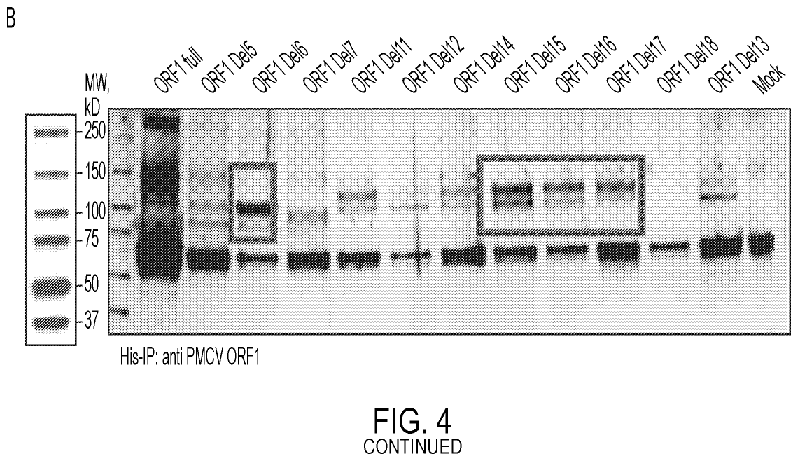

The results can be summarized as follows: After the first round (200-amino-acid-long deletions) expression at the cell membrane was achieved only with antigen from constructs IntDel 4-7. Accordingly, the N-terminal portion is required for routing the antigen to the cell membranes. Constructs made in the second round (50-amino-acid-long deletions), focused therefore on the region covered by IntDel 4-7. All these constructs successfully expressed antigen at the cell membrane, except that cell surface expression was lower for IntDel 18 (the construct lacking the amino acid sequence set forth in SEQ ID NO: 15). The deletion constructs (IntDel 15-17) resulted in increased membrane expression levels. IntDel 6 (the construct lacking amino acids 500-699 of SEQ ID NO: 1) from the first round was also found to have higher expression levels. Except for IntDel 42 (the construct lacking amino acids set forth in SEQ ID NO: 14) doing slightly better than the others, no further candidates were revealed in the third round. The results are illustrated in FIG. 4.

Based on combined results from evaluation of immunofluorescence staining and Western blotting, several deletion candidates have been selected based on results from several vaccine trials in the project. For all vaccines in the first study the antigen encoding genes were inserted downstream of a CMV promoter in the eukaryotic expression vector NTC9385R (Nature Technology Corporation). All plasmids were diluted in sterile phosphate-buffered saline (PBS) to 10 μg per 50 μl injection volume. All fish received one intramuscular injection on each side of the fish and a total dose of 20 μg. A non-immunized control group was included and received 2×50 μl PBS, as summarized in Table 7.

TABLE 7

| | | Description plasmid | | Final | |
|---|---|---|---|---|---|
| | | encoding antigen | | conc | |
| Vaccine | | (positions relates to nucleotide | Plasmid | construct | Dose ug |
| group | Description | position PMCV ORF1) | backbone | (μg/ml) | (2 × 0.05 ml) |
| 1 | G-ORF1 | G-PMCV_ORF1_full_1-2583 | NTC9385R | 200 | 20 |
| 2 | G-IntDel-6 | G-PMCV ORF1_IntDel6 | NTC9385R | 200 | 20 |
| | | (pos 1798-2397) | | | |
| 3 | G-IntDel-15 | G-PMCV ORF1_IntDel15 | NTC9385R | 200 | 20 |
| | | (pos 1984-2133) | | | |
| 4 | G-IntDel-16 | G-PMCV ORF1_IntDel16 | NTC9385R | 200 | 20 |
| | | (pos 2134-2283) | | | |
| 5 | G-IntDel-17 | G-PMCV ORF1_IntDel17 | NTC9385R | 200 | 20 |
| | | (pos 2284-2433) | | | |
| 6 | PBS | | NA | | |

Atlantic salmon (Salmo salar) (n=30 per group) kept in freshwater and with average weight of 20 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 48 days at 12° C. (light:dark 12:12) before the fish were anaesthetized again using Tricain (PHARMAQ) and challenged with infectious PMCV by intraperitoneal injection of 0.1 ml of homogenized heart (isolate ID AL V1273 Heart) originating from a Norwegian field outbreak of CMS. The fish (n=15 per group, per sampling-point) were then sampled at two timepoints; 19 days and 49 days post challenge. Heart ventricle and kidney were collected on RNALATER® from all sampled fish for subsequent RNA extraction. Hearts (ventricle and atrium) were also fixed in formalin from all fish on the last sampling point (day 49) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 3 according to severity, where 0 indicates no pathologic finding and 3 indicate presence of severe pathology (service provided by PHARMAQ Analytiq, Norway).

The results for viral load at 3 weeks post challenge and histoscore at 7 weeks post challenge are shown in Tables 8 and 9, respectively. The combined data of all results showed that the IntDel variants 15, 16 and 17 (wherein the antigen lacked the amino acid sequences set forth in SEQ ID NOs 3, 4, or 5, respectively) performed better than full-length capsid protein (G-ORF1).

TABLE 8
Viral RNA in kidney 3 weeks post challenge.

| | | Mean | Standard | | p-value |
|---|---|---|---|---|---|
| Vaccine | | Ct-value, | deviation, | | (compared |
| group | Description | PMCV | Ct | N | to PBS) |
| 6 | PBS | 20.61 | 1.49 | 15 | NA |
| 1 | G-ORF1 | 23.77 | 2.57 | 15 | 0.0002 |
| 2 | G-IntDel-6 | 21.23 | 1.41 | 15 | 0.86 |
| 3 | G-IntDel-15 | 23.13 | 2.81 | 15 | 0.0033 |
| 4 | G-IntDel-16 | 22.92 | 1.20 | 15 | 0.0082 |
| 5 | G-IntDel-17 | 23.13 | 1.71 | 15 | 0.0032 |

The indicated p-value was calculated by comparing individual groups with the PBS control group using a One-way ANOVA, with Dunnett's post test.
NA = Not applicable.

TABLE 9
Histopathological lesions (histoscore) in atrium 7 weeks post challenge.

| | | Mean | Standard | | p-value |
|---|---|---|---|---|---|
| Vaccine- | | histoscore, | deviation, | | (compared |
| group | Description | Atrium | histoscore | N | to PBS) |
| 6 | PBS | 1.75 | 0.84 | 15 | NA |
| 1 | G-ORF1 | 1.60 | 0.83 | 15 | 0.99 |
| 2 | G-IntDel-6 | 1.54 | 0.99 | 15 | 0.95 |
| 3 | G-IntDel-15 | 0.75 | 0.75 | 15 | 0.012 |
| 4 | G-IntDel-16 | 1.10 | 0.63 | 15 | 0.16 |
| 5 | G-IntDel-17 | 1.32 | 0.87 | 15 | 0.53 |

The indicated p-value was calculated by comparing individual groups with the PBS control group using a One-way ANOVA, with Dunnett's post test.
NA = Not applicable.

An additional clinical trial was performed to evaluate three new antigen deletion candidates. For all vaccines in the first study the antigen encoding genes were inserted downstream of a CMV promoter in the eukaryotic expression vector NTC9385R (Nature Technology Corporation). All plasmids were diluted in sterile phosphate-buffered saline (PBS) to 10 μg per 50 μl injection volume. All fish received one intramuscular injection on each side of the fish and a total dose of 20 μg. A non-immunized control group was included and received 2×50 μl PBS, as summarized in Table 10.

TABLE 10

| | | Description plasmid | | Final | |
|---|---|---|---|---|---|
| | | encoding antigen | | conc | |
| Vaccine | | (positions relates to nucleotide | Plasmid | construct | Dose ug |
| group | Description | position PMCV ORF1) | backbone | (μg/ml) | (2 × 0.05 ml) |
| 1 | G-ORF1 | G-PMCV_ORF1_full_1-2583 | NTC9385R | 200 | 20 |
| 2 | G-IntDel-5 | G-PMCV ORF1_IntDel5 | NTC9385R | 200 | 20 |
| | | (pos 1498-2097) | | | |
| 3 | G-IntDel-7 | G-PMCV ORF1_IntDel7 | NTC9385R | 200 | 20 |
| | | (pos 2098-2583) | | | |
| 4 | G-IntDel-42 | G-PMCV ORF1_IntDel42 | NTC9385R | 200 | 20 |
| | | (pos 2440-2469) | | | |
| 5 | PBS | | NA | | |

Atlantic salmon (Salmo salar) (n=30 per group) kept in one tank of freshwater and with average weight of 28 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 58 days at 12° C. (light:dark 12:12) before the fish were anaesthetized again using Tricain (PHARMAQ) and challenged with infectious PMCV by intraperitoneal injection of 0.1 ml of homogenized heart (isolate ID AL V1289) originating from a Norwegian field outbreak of CMS. The fish (n=15 per group) were then sampled at two timepoints; 3 weeks and 7 weeks post challenge. Heart ventricle and kidney were collected on RNALATER© from all sampled fish for subsequent RNA extraction. Hearts (ventricle and atrium) were also fixed in formalin from all fish on the last sampling point (week 7) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 4 according to severity, where 0 indicates no pathologic finding and 4 indicate presence of severe pathology (service provided by PHARMAQ Analytiq, Norway).

The results for week 3 and week 7 are summarized in Tables 11 and 12, respectively.

TABLE 11
Viral RNA in kidney 3 weeks post challenge. The indicated p-value
was calculated by comparing individual groups with the PBS control
group using a One-way ANOVA, with Dunnett's
post test. NA = Not applicable.

| | | | | | p-value |
|---|---|---|---|---|---|
| Vaccine | | Mean Ct- | Standard | | (compared to |
| group | Description | value, PMCV | deviation, Ct | N | PBS) |
| 5 | PBS | 19.44 | 1.13 | 15 | NA |
| 1 | G-ORF1 | 20.48 | 1.91 | 15 | 0.38 |
| 2 | G-Del5 | 22.58 | 2.69 | 15 | <0.0001 |
| 3 | G-Del7 | 21.12 | 1.99 | 15 | 0.06 |
| 4 | G-Del42 | 21.12 | 1.40 | 15 | 0.06 |

TABLE 12
Histopathological lesions (histoscore) in atrium 7 weeks post challenge.
The indicated p-value was calculated by comparing individual groups
with the PBS control group using a One-way ANOVA, with Dunnett's
post test. NA = Not applicable.

| | | Mean | Standard | | p-value |
|---|---|---|---|---|---|
| Vaccine- | | histoscore, | deviation, | | (compared to |
| group | Description | Atrium | histoscore | N | PBS) |
| 5 | PBS | 2.14 | 0.36 | 14 | NA |
| 1 | G-ORF1 | 2.07 | 0.70 | 15 | 0.99 |
| 2 | G-Del5 | 1.64 | 1.15 | 14 | 0.28 |
| 3 | G-Del7 | 1.80 | 0.56 | 15 | 0.59 |
| 4 | G-Del42 | 1.20 | 0.94 | 15 | 0.0075 |

### Example 3: Vaccine Backbone Selection

To optimize vaccine efficacy, properties of the plasmid backbone such as vector size and functional elements are also important. Three vector candidates were compared with regards to in vitro antigen expression. The candidates included pcDNA3.1, pVAX1 (INVITROGEN™), NTC9385R (Nature Technology Corporation), and two variants of NTC9385R that co-express innate immunostimulatory elements: a RIG-1 agonist that function as a type I interferon inducing adjuvant (NTC9385R-eRNA41H) and the RIG-1 agonist as well as a Toll-like receptor 9 stimulating CpG motif (NTC9385R-eRNA41H-CpG). The NTC9385R NANOPLASMID™ is smaller in size than the other traditional vectors, allowing for improved uptake and persistence in transfected cells and harbors a modified promoter claimed to enhance antigen expression. It contains an RNA-based sucrose selection antibiotic free marker (RNA-OUT) that replaces the use of antibiotics in the production process and a modified replication origin that secures that the plasmid can only replicate in a specific E. coli production strain which is beneficial from a safety perspective. The smaller size can affect transfection efficiency and level and duration of expression. Additionally, some bacterial region protein marker genes like resistance marker genes have been shown to dramatically reduce vector expression. Bacterial regions larger than 1 kilobase have been shown to silence transgene expression in quiescent tissue such as the liver, likely due to untranscribed bacterial region mediated heterochromatin formation that spreads to the eukaryotic region and inactivates the promote (Suschak et al., 2017).

Evaluating in vitro expression levels of the vaccine antigen G-ORF1 in the five different plasmid backbones by Western blotting was done as follows: CHH-1 cells were trypsinated and seeded into 6-well plates (1 000 000 cells per well and 3 ml cell-medium). The cells were incubated at 15° C. ON in a CO₂ incubator (5% CO₂). The cells were transfected the day after seeding using 2.5 μg plasmid/well and LIPOFECTAMIN® 3000 (Thermo Fisher Scientific) according manufacturer's instructions. Cells were put back in sealed bags and incubated at 15° C. in a CO₂ incubator (5% CO₂) for five days before further analysis. Plates were then placed on ice and medium removed. Cells were first washed twice with ice-cold PBS. Total cell lysates were prepared by adding 150 μl NP40 lysis buffer (Invitrogen, #FNN021) with 1× Protease inhibitor cocktail (Roche, #1697498) directly to the wells. Lysates were transferred to pre-cooled Eppendorf tubes, incubated for 30 min at 4° C. while re-suspending cells by flicking the tube occasionally, centrifuged at 12 000 rpm for 10 min at 4° C. and the supernatant transferred to fresh tubes and kept on ice until immunoprecipitation. On ice, 1 μg of mouse IgG1anti 6×-His monoclonal antibody 4E3D10H2/E3 (Fisher Scientific #15442890, 1 mg/ml) was added to each sample and incubated for 4° C. overnight on a rotator. Protein G-coupled beads were prepared (25 μl/sample) by washing the immobilized beads 3-5× with ˜1 ml cold PBST and re-suspended in 25 μl of PBST with protease inhibitors per aliquot. On ice, 25 μl of pre-equilibrated beads were added to each sample and the sample-beads mixture incubated for 1 hour at RT with rotation. The beads were washed with 500 μl of ice cold PBST with proteinase inhibitors 3-5 times to remove non-specific binding with gentle mixing of the beads between each wash. After the last wash, as much buffer as possible was removed from the beads and 40 μl of 1× Laemmli Sample Buffer (BioRad #161-0737) added (prepared by mixing 20 μl 2× Laemmli Sample Buffer with 2 μl β-mercaptoethanol and 18 μl dH2O). Samples were incubated at 70° C. for 10 minutes, quickly centrifuged and magnetized, and eluent moved to a new vial. Before loading on a 4-20% CRITERION™ TGX STAIN-FREE™ Gel, all samples were denatured at 98° C. for 5 min. The gel was run for approximately 45 min at 200V and blotted using the TRANS-BLOT® TURBO™ Transfer System (BioRad), and TRANS-BLOT® TURBO™ Midi PVDF Transfer Packs #170-4157. 7 minutes turbo program was used. The membrane was immediately put in blocking-buffer (5% skimmed milk in PBST). Blocking was performed for 1 hour at room-temperature. The blot was incubated with primary antibody rabbit anti-delta PMCV ORF1 diluted 1: 500 in 1% skimmed milk/PBST over-night at 4° C. in rotator. The following day the blots were washed 2×15 minutes with PBST and incubated with secondary antibody polyclonal swine anti-rabbit (DAKO #P0217) immunoglobulin HRP diluted 1:1000 in 2% skimmed milk and PRECISION PROTEIN™ StrepTactin-HRP Conjugate 1:10000 (to visualize ladder) for 1 hour at RT. The blot was washed 2×15 minutes with PBST before detection of signal by Clarity western ECL substrate (BioRad #170-5060).

For all vaccines in the present study the full-length PMCV ORF1 capsid gene routed for expression at the cell membrane (G-ORF1), was inserted downstream of a CMV promoter in different eukaryotic expression vectors according to the table below. All plasmids were diluted in sterile phosphate-buffered saline (PBS) to 10 μg per 50 μl injection volume. All fish received one intramuscular injection on each side of the fish and a total dose of 20 μg. A non-immunized control group was included and received 2×50 μl PBS, as summarized in table 13.

TABLE 13

| | | | | Final | |
|---|---|---|---|---|---|
| | | | | conc | Dose μg |
| Vaccine | | Description plasmid | | construct | (2 × 0.05 |
| group | Description | encoding antigen | Plasmid backbone | (μg/ml) | ml) |
| 1 | G-ORF1-pcDNA3.1 | G- | pcDNA3.1(+) | 200 | 20 |
| | | PMCV_ORF1_full_1- | | | |
| | | 2583 | | | |
| 2 | G-ORF1-pVAX1 | G- | pVAX1 | 200 | 20 |
| | | PMCV_ORF1_full_1- | | | |
| | | 2583 | | | |
| 3 | G-ORF1-NTC9385R | G- | NTC9385R | 200 | 20 |
| | | PMCV_ORF1_full_1- | | | |
| | | 2583 | | | |
| 4 | G-ORF1- | G- | NTC9385R- | 200 | 20 |
| | NTC9385R- | PMCV_ORF1_full_1- | eRNA41H-CpG | | |
| | eRNA41H-CpG | 2583 | | | |
| 5 | G-ORF1- | G- | NTC9385R- | 200 | 20 |
| | NTC9385R- | PMCV_ORF1_full_1- | eRNA41H | | |
| | eRNA41H | 2583 | | | |
| 6 | PBS | | NA | | |

Atlantic salmon (Salmo salar) (n=30 per group) kept in freshwater and with average weight of 29 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 59 days at 12° C. (light:dark 12:12) before the fish were anaesthetized again using Tricain (PHARMAQ) and challenged with infectious PMCV by intraperitoneal injection of 0.1 ml of homogenized heart (isolate ID AL V1273 Heart) originating from a Norwegian field outbreak of CMS. The fish (n=15 per group) were then sampled at two timepoints; 20 days and 48 days post challenge. Heart ventricle and kidney were collected on RNALATER® from all sampled fish for subsequent RNA extraction. Hearts (ventricle and atrium) were also fixed in formalin from all fish on the last sampling point (day 48) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 3 according to severity, where 0 indicates no pathologic finding and 3 indicate presence of severe pathology (service provided by PHARMAQ Analytiq, Norway).

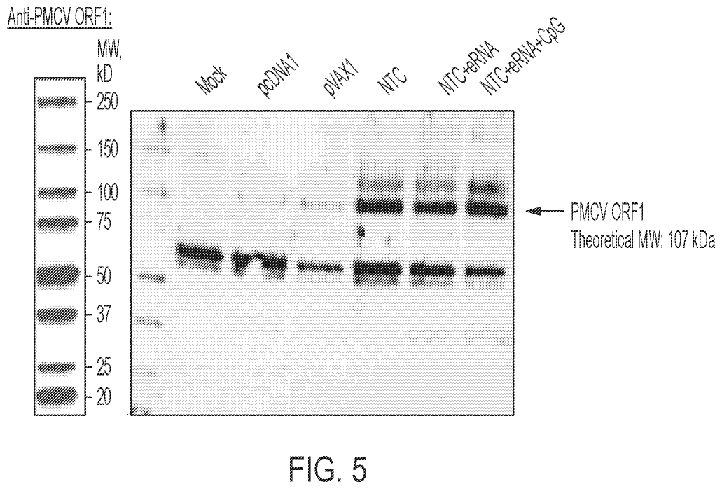

Western blot analysis of transfected CHH-1 cells demonstrated that all three NTC9385R plasmid variants mediated superior antigen expression compared to the pcDNA3.1 and pVAX1 plasmid backbone (FIG. 5). Clinical trials showed at least as good protection for NTC9385R-based vaccines as those based on pcDNA3.1/pVAX1 (See Table 14 and Table 15 for three and seven weeks, respectively). The combined dataset showed a trend towards superior protection by NTC9385R-based vaccine backbones. The NTC9385R plasmids with immunostimulatory elements did not provide additional effect.

TABLE 14
Viral RNA in kidney 3 weeks post challenge. The indicated p-value was
calculated by comparing individual groups with the PBS control group using
a One-way ANOVA, with Dunnett's post test. NA = Not applicable.

| | | Mean Ct- | Standard | | p-value |
|---|---|---|---|---|---|
| Vaccine | | value, | deviation, | | (compared to |
| group | Description | PMCV | Ct | n | PBS) |
| 6 | PBS | 21.73 | 1.25 | 15 | NA |
| 1 | G-ORF1-pcDNA3.1 | 22.66 | 1.20 | 15 | 0.24 |
| 2 | G-ORF1-pVAX1 | 22.57 | 1.51 | 15 | 0.35 |
| 3 | G-ORF1-NTC9385R | 23.78 | 1.71 | 15 | 0.0008 |
| 4 | G-ORF1-NTC9385R-eRNA41H-CpG | 23.52 | 1.42 | 15 | 0.0034 |
| 5 | G-ORF1-NTC9385R-eRNA41H | 23.03 | 1.28 | 15 | 0.053 |

TABLE 15
Viral RNA in kidney 7 weeks post challenge. The indicated p-value was
calculated by comparing individual groups with the PBS control group
using a One-way ANOVA, with Dunnett's post test. NA = Not applicable.

| | | Mean Ct- | Standard | | p-value |
|---|---|---|---|---|---|
| Vaccine | | value, | deviation, | | (compared to |
| group | Description | PMCV | Ct | n | PBS) |
| 6 | PBS | 22.45 | 1.53 | 15 | NA |
| 1 | G-ORF1-pcDNA3.1 | 23.75 | 1.73 | 15 | 0.26 |
| 2 | G-ORF1-pVAX1 | 23.77 | 2.01 | 15 | 0.23 |
| 3 | G-ORF1-NTC9385R | 23.94 | 2.42 | 15 | 0.14 |
| 4 | G-ORF1-NTC9385R-eRNA41H-CpG | 25.63 | 1.77 | 15 | 0.0001 |
| 5 | G-ORF1-NTC9385R-eRNA41H | 24.44 | 2.02 | 15 | 0.031 |

### Example 4: Adjuvant Selection

Molecular adjuvants show great promise for both increasing immunogenicity and extending the longevity of the immune response, and these molecular adjuvants expressing cytokines, chemokines, or co-stimulatory molecules may be co-administered with the DNA vaccine plasmid encoding the antigen. Cells transfected by molecular adjuvant plasmids secrete the adjuvant into the surrounding region, stimulating local antigen presenting cells (Suschak et al., 2017). Adjuvant activity of fish type I interferons have already been shown in a virus DNA vaccination model for infectious salmon anemia virus (ISAV) in Atlantic salmon (Chang et al., 2015). In the paper, it was demonstrated that Type I IFNs enhanced the antibody response against ISAV-hemagglutinin protein and provided increased protection against viral challenge.

The following molecular adjuvants: IFNa, IFNb, IFNc, IFNd, IFNy, IL-2, IL-4, IL-12 and VHSV-G were tested in vivo together with the full-length PMCV ORF1 vaccine antigen (G-ORF1) for adjuvant activity using the high-throughput model for rapid efficacy screening of candidates.

This model is based on a surprising discovery that that differences in histopathological lesions 50 days post challenge could be predicted by viral RNA levels in heart and kidney in the same groups 20 days post challenge. Therefore, for rapid screening of many groups, only one sampling point 3 weeks post challenge needs to be used.

For all groups in the in vivo screening study of adjuvants, the vaccine antigen and plasmid encoded adjuvants were administered on separate plasmids. The gene encoding the antigen (G-ORF1) and various plasmid encoded adjuvant genes were all inserted downstream of a CMV promoter in the eukaryotic expression vector pcDNA3.1(+) (Invitrogen). Plasmids were diluted in sterile phosphate-buffered saline (PBS) to 10 μg per 50 μl injection volume according to the table below. All fish received one intramuscular injection on each side of the fish and a total dose of 20 μg plasmid (20 μg of each plasmid if given antigen plus adjuvant). Two non-immunized control groups were included, one group received 20 μg of a control vaccine (eGFP in pcDNA3.1) and one group received 2×50 μl PBS, as summarized in Table 16.

Atlantic salmon (Salmo salar) (n=15 per group) kept in freshwater and with average weight of 31 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 50 days at 12° C. (light:dark 12:12) before the fish were anaesthetized again using Tricain (PHARMAQ) and challenged with infectious PMCV by intraperitoneal injection of 0.1 ml of homogenized heart (isolate ID ALV1223 Heart) originating from a Norwegian field outbreak of CMS. The fish (n=15 per group) were then sampled 21 days post challenge. Heart ventricle and kidney was collected on RNALATER© from all sampled fish for subsequent RNA extraction. RNA was extracted from all samples stored on RNALATER® and analyzed by Real-Time RT-PCR for presence of PMCV RNA using a commercially available service from PHARMAQAnalytiq (Norway).

Following 7 weeks of immunization and 3 weeks of challenge, the model found that while most of the adjuvant strategies failed in improving efficacy, one adjuvant—IFNb significantly increased the level of effect. The results are listed in Table 17.

TABLE 16

| | | | | | | Final | Dose |
|---|---|---|---|---|---|---|---|
| | | Detailed | | Detailed | | conc | μg |
| | | description | | description | Final conc | construct | (2 × |
| | Description | construct A | Description | construct B | construct | B | 0.05 |
| group | (Antigen) | (Antigen) | (Adjuvant) | (Adjuvant) | A (μg/ml) | (μg/ml) | ml) |
| 1 | G-ORF1 | G- | None | | 200 | | 20 |
| | | PMCV_ORF1_full_{—} | | | | | |
| | | 1-2583 | | | | | |
| 2 | eGFP | enhanced Green | None | | 200 | | 20 |
| | | Fluorescent | | | | | |
| | | Protein (control) | | | | | |
| 3 | G-ORF1 | G- | IFNa1 | IFNa1 | 200 | 200 | 20 |
| | | PMCV_ORF1_full | | adjuvant | | | each |
| | | 1-2583 | | construct | | | |
| 4 | G-ORF1 | G- | IFNb | IFNb | 200 | 200 | 20 |
| | | PMCV_ORF1_full_{—} | | adjuvant | | | each |
| | | 1-2583 | | construct | | | |
| 5 | G-ORF1 | G- | IFNc | IFNc | 200 | 200 | 20 |
| | | PMCV_ORF1_full_{—} | | adjuvant | | | each |
| | | 1-2583 | | construct | | | |
| 6 | G-ORF1 | G- | IFNd | IFNd | 200 | 200 | 20 |
| | | PMCV_ORF1_full_{—} | | adjuvant | | | each |
| | | 1-2583 | | construct | | | |
| 7 | G-ORF1 | G- | Interleukin | IL-2 | 200 | 200 | 20 |
| | | PMCV_ORF1_full_{—} | 2 | adjuvant | | | each |
| | | 1-2583 | | construct | | | |
| 8 | G-ORF1 | G- | Interleukin | IL-4 | 200 | 200 | 20 |
| | | PMCV_ORF1_full_{—} | 4 | adjuvant | | | each |
| | | 1-2583 | | construct | | | |
| 9 | G-ORF1 | G- | Interleukin | IL-12 | 200 | 200 | 20 |
| | | PMCV_ORF1_full_{—} | 12 | adjuvant | | | each |
| | | 1-2583 | | construct | | | |
| 10 | G-ORF1 | G- | IFNg | IFN gamma | 200 | 200 | 20 |
| | | PMCV_ORF1_full_{—} | | adjuvant | | | each |
| | | 1-2583 | | construct | | | |
| 11 | G-ORF1 | G- | VHSV-G | VHSV-g | 200 | 200 | 20 |
| | | PMCV_ORF1_full_{—} | | protein | | | each |
| | | 1-2583 | | DNA | | | |
| | | | | vaccine | | | |
| 12 | G-ORF1 | G- | rORF1 | Rec. protein | 200 | NA | 20 |
| | | PMCV_ORF1_full_{—} | (PMCV) | PMCV ORF1 | | | |
| | | 1-2583 | | (E. coli)* | | | |
| 13 | PBS | PBS control | | | | | |

TABLE 17
Viral RNA in kidney 3 weeks post challenge. The indicated p-value was
calculated by comparing individual groups with the G-ORF1 control group
(no adjuvant) using a One-way ANOVA, with Dunnett's post test.

| | | | Mean Ct- | Standard | | p-value (compared |
|---|---|---|---|---|---|---|
| Vaccine | Antigen | | value, | deviation, | | to G-ORF1 alone - |
| group | construct | Adjuvant | PMCV | Ct | n | group 1) |
| 13 | PBS | None | 19.49 | 0.96 | 14 | 0.0002 |
| 1 | G-ORF1 | None | 23.12 | 2.60 | 31 | NA |
| 2 | eGFP | None | 22.04 | 1.51 | 16 | 0.73 |
| 3 | G-ORF1 | IFNa1 | 23.17 | 3.29 | 15 | 0.99 |
| 4 | G-ORF1 | IFNb | 26.06 | 3.05 | 15 | 0.0033 |
| 5 | G-ORF1 | IFNc | 23.53 | 3.27 | 14 | 0.99 |
| 6 | G-ORF1 | IFNd | 21.82 | 2.50 | 16 | 0.52 |
| 10 | G-ORF1 | IFNg | 21.09 | 1.69 | 14 | 0.11 |
| 7 | G-ORF1 | Interleukin 2 | 20.70 | 3.20 | 13 | 0.04 |
| 8 | G-ORF1 | Interleukin 4 | 21.72 | 2.01 | 15 | 0.45 |
| 9 | G-ORF1 | Interleukin 12 | 23.03 | 3.24 | 16 | 0.99 |
| 11 | G-ORF1 | VHSV-G | 20.25 | 2.52 | 15 | 0.004 |
| 12 | G-ORF1 | rORF1 (PMCV) | 19.42 | 0.74 | 10 | 0.001 |

NA = Not applicable.

IFNb and IFNc were selected for a follow up fish trial. For all groups in this trial, the vaccine antigen and plasmid encoded adjuvants were administered on separate plasmids. The gene encoding the antigen (G-ORF1) and the plasmid encoded adjuvant genes (IFNb or IFNc) were all inserted downstream of a CMV promoter in the eukaryotic expression vector pcDNA3.1(+) (Invitrogen). Plasmids were diluted in sterile phosphate-buffered saline (PBS) to 10 μg per 50 μl injection volume according to the table below. All fish received one intramuscular injection on each side of the fish and a total dose of 20 μg plasmid (20 μg of each plasmid if given antigen plus adjuvant). A non-immunized control group was included and received 2×50 μl PBS, as summarized in Table 18.

TABLE 18

| | | | Detailed | Detailed | | | |
|---|---|---|---|---|---|---|---|
| | | | description | description | Final conc | Final conc | Dose μg |
| | Description | Description | construct A | construct B | construct | construct | (2 × 0.05 |
| Group | (Antigen) | (Adjuvant) | (Antigen) | (Adjuvant) | A (μg/ml) | B (μg/ml) | ml) |
| 1 | G-ORF1 | None | G- | | 200 | | 20 |
| | | | PMCV_ORF1_{—} | | | | |
| | | | full_1-2583 | | | | |
| 2 | G-ORF1 | IFNb | G- | IFNb | 200 | 200 | 20 each |
| | | | PMCV_ORF1_{—} | adjuvant | | | |
| | | | full_1-2583 | construct | | | |
| 3 | None | IFNb | | IFNb | | 200 | 20 |
| | | | | adjuvant | | | |
| | | | | construct | | | |
| 4 | G-ORF1 | IFNc | G- | IFNc | 200 | 200 | 20 each |
| | | | PMCV_ORF1_{—} | adjuvant | | | |
| | | | full_1-2583 | construct | | | |
| 5 | None | IFNc | | IFNc | | 200 | 20 |
| | | | | adjuvant | | | |
| | | | | construct | | | |
| 6 | PBS | | PBS control | | | | |

Atlantic salmon (Salmo salar) (n=30 per group) kept in freshwater and with average weight of 29 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 59 days at 12° C. (light:dark 12:12) before the fish were anaesthetized again using Tricain (PHARMAQ) and challenged with infectious PMCV by intraperitoneal injection of 0.1 ml of homogenized heart (isolate ID ALV1273 Heart) originating from a Norwegian field outbreak of CMS. The fish (n=15 per group) were then sampled at two timepoints; 20 days and 48 days post challenge. Heart ventricle and kidney were collected on RNALATER® from all sampled fish for subsequent RNA extraction. Hearts (ventricle and atrium) were also fixed in formalin from all fish on the last sampling point (day 48) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 3 according to severity, where 0 indicates no pathologic finding and 3 indicate presence of severe pathology (service provided by PHARMAQ Analytiq, Norway).

The results showed that IFNb was superior to IFNc (Table 19). The difference between the two adjuvants was most profound at the 7 weeks post challenge sampling point (data from earlier time point not shown). The group receiving adjuvant only was also partly protected reflecting that IFNs are critical effectors of both innate and adaptive immune responses.

TABLE 19
Viral RNA in heart 7 weeks post challenge. The indicated p-value
was calculated by comparing individual groups with the PBS control
group using a One-way ANOVA, with Dunnett's post test.

| | | | | | | p-value |
|---|---|---|---|---|---|---|
| | | | Mean Ct- | Standard | | (compared |
| Vaccine | | | value, | deviation, | | to |
| group | Antigen | Adjuvant | PMCV | Ct | n | PBS) |
| 6 | PBS | PBS | 23.17 | 2.40 | 15 | NA |
| 1 | G-ORF1 | PBS | 25.64 | 2.61 | 15 | 0.40 |
| 2 | G-ORF1 | IFNb | 30.77 | 5.61 | 15 | 0.0001 |
| 3 | None | IFNb | 30.38 | 4.40 | 15 | 0.0002 |
| 4 | G-ORF1 | IFNc | 25.16 | 4.79 | 15 | 0.64 |
| 5 | None | IFNc | 22.48 | 2.15 | 15 | 0.99 |

NA = Not applicable.

### Example 5—Adjuvant Delivery

The aim of this experiment was to determine the best way to deliver the antigen and the molecular adjuvant.

Plasmid-encoded adjuvants can be delivered either on a separate plasmid or included on the same plasmid as the one encoding the antigen. If expressed from the same plasmid as the antigen, this can e.g. be done in the following ways:
1. From a separate but identical promoter as the vaccine antigen.
2. From a separate similar (but not identical) promoter as the vaccine antigen.
3. As a fusion protein with the antigen—separated by a P2A peptide sequence to facilitate self-cleavage.

In this example, different delivery options for the adjuvant were tested. The cell surface expressed PMCV ORF1 antigen (G-ORF1) was given to all groups. The adjuvant was provided in the following ways:
1. On a separate plasmid (G-ORF1+IFNb)
2. Expressed together with the antigen, downstream of the same CMV promoter and separated from the antigen by a self-cleavage 2A peptide (G-ORF1-P2A-IFNb)
3. Expressed on the same plasmid as the antigen but downstream of a second CMV promoter (G-ORF1-CMV-IFNb),
4. One group did not receive the adjuvant only the antigen (G-ORF1).
5. A non-vaccinated control group receiving PBS was also included.

For all vaccines in the present study the genes were inserted downstream of a CMV promoter in the eukaryotic expression vector pVAX1 (Invitrogen). The antigen used was G-ORF1 (G-PMCV_ORF1_full_1-2583). All vaccines were diluted in sterile phosphate-buffered saline (PBS) to 10 μg per 50 μl injection volume. All fish received one intramuscular injection on each side of the fish and a total dose of 20 μg. For fish receiving the antigen and the plasmid encoded adjuvant on separate plasmids, the dose for each plasmid was 20 μg. A non-immunized control group was included and received 2×50 μl PBS. In addition, one group received a codon-changed version of the plasmid encoded adjuvant, as summarized in Table 20.

TABLE 20

| | | | | Dose μg |
|---|---|---|---|---|
| Group | Construct 1 | Construct 2 | Adjuvant administration | (2 × 0.05 ml) |
| 1 | G-ORF1 | IFNb codon | IFNb adjuvant codon changed | 20 each |
| | | changed | provided on separate plasmid | |
| 2 | G-ORF1 | IFNb | IFNb adjuvant provided on | 20 each |
| | | | separate plasmid | |
| 3 | G-ORF1-P2A-IFNb | | IFNb provided on same plasmid | 20 |
| | | | and under same promoter as | |
| | | | antigen | |
| 4 | G-ORF1-CMV-IFNb | | IFNb provided on same plasmid | 20 |
| | | | but downstream of a second | |
| | | | CMV promoter | |
| 5 | G-ORF1 | | No adjuvant control group | 20 |
| 6 | PBS | | | |

Atlantic salmon (Salmo salar) (n=30 per group) kept in freshwater and with average weight of 25 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 49 days at 12° C. (light:dark 12:12) before the fish were anaesthetized again using Tricain (PHARMAQ) and challenged with infectious PMCV by intraperitoneal injection of 0.1 ml of homogenized heart (isolate ID ALV1273 Heart) originating from a Norwegian field outbreak of CMS. The fish (n=15 per group) were then sampled at two timepoints; 19 days and 47 days post challenge. Heart ventricle and kidney were collected on RNALATER© from all sampled fish for subsequent RNA extraction. Hearts (ventricle and atrium) were also fixed in formalin from all fish on the last sampling point (day 47) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 3 according to severity, where 0 indicates no pathologic finding and 3 indicate presence of severe pathology (service provided by PHARMAQ Analytiq, Norway).

The results from the clinical trial are exemplified by histoscore data from heart at 7 weeks post challenge (Table 21).

TABLE 21
Histopathological lesions (histoscore) in atrium 7 weeks post challenge. The indicated
p-value was calculated by comparing individual groups with the PBS control group
using a One-way ANOVA, with Dunnett's post test. NA = Not applicable.

| | | Mean | Standard | | p-value |
|---|---|---|---|---|---|
| Vaccine- | | histoscore, | deviation, | | (compared |
| group | Description | Atrium | histoscore | N | to PBS) |
| 6 | PBS | 2.46 | 0.54 | 15 | NA |
| 5 | G-ORF1 | 1.73 | 1.00 | 15 | 0.20 |
| 2 | G-ORF1 + IFNb | 1.00 | 0.90 | 15 | 0.0014 |
| 4 | G-ORF1-CMV-IFNb | 1.13 | 1.03 | 15 | 0.0035 |
| 3 | G-ORF1-P2A-IFNb | 1.73 | 1.22 | 15 | 0.20 |
| 1 | G-ORF1-CMV-IFNb_codon changed | 1.04 | 0.96 | 15 | 0.003 |

Antigen expression levels and adjuvant activity for the different constructs have also been assessed in vitro (results not shown). The combined results suggest that the preferred approach is to provide the adjuvant on the same plasmid as the antigen under an identical but separate promoter as the antigen but delivery on different plasmids can also be effective.

### Example 6—Duration of Vaccine Efficiency

The initial studies showed that fish receiving adjuvant only were partly protected, reflecting that IFNs are critical effectors of both innate and adaptive immune responses. To study the duration of protection obtained by including a molecular adjuvant in a DNA construct, a long-term follow-up vaccine trial was carried out.

For all vaccines in the present study the genes were inserted downstream of a CMV promoter in eukaryotic expression vectors from Nature Technology Corporation (NTC). The backbones were either NTC9385R or NTC9385R-eRNA41H-CpG. For the group receiving both an antigen and the plasmid encoded adjuvant, these were either provided on separate plasmids (20 μg each plasmid) or on the same plasmid (20 μg). All fish received one intramuscular injection on each side of the fish (0.05 ml). A non-immunized control group was included and received 2×50 μl PBS. For details about vaccine groups, see Table 22 below.

TABLE 22

| | | Backbone | | Backbone | | |
|---|---|---|---|---|---|---|
| | Construct | construct | Construct | construct | | Dose μg |
| Group | 1 | 1 | 2 | 2 | Adjuvant administration | (2 × 0.05 ml) |
| 1 | G-ORF1 | NTC9385R | IFNb | NTC9385R | Antigen + adjuvant - on | 20 each |
| | | | | | two separate plasmids | |
| 2 | G-ORF1 | NTC9385R | | | Antigen only | 20 |
| 3 | IFNb | NTC9385R | | | Adjuvant only | 20 |
| 4 | G-ORF1- | NTC9385R | | | Antigen + adjuvant on | 20 |
| | CMV- | | | | same plasmid | |
| | IFNb | | | | | |
| 5 | G-ORF1- | NTC9385R- | | | Antigen + adjuvant on | 20 |
| | CMV- | eRNA41H- | | | same plasmid | |
| | IFNb | CpG | | | | |
| 6 | PBS | | | | | |

Atlantic salmon (Salmo salar) (n=60 per group) kept in freshwater and with average weight of 17 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. The fish were then kept in a reservoir tank where immunity was allowed to develop for 78 days, 120 days or 183 days at 12C (light:dark 12:12). At each of these time-points cohorts of fish (n=15 per group on time-points 78 dpv and 183 dpv, and n=30 per group on time-point 120 dpv) were moved to a challenge tank. They were anaesthetized again using Tricain (PHARMAQ) and challenged with infectious PMCV by intraperitoneal injection of 0.1 ml of homogenized heart (isolate ID ALV1273 Heart) originating from a Norwegian field outbreak of CMS. The fish (n=15 per group) were then sampled 3 weeks post challenge. Heart ventricle and kidney were collected on RNALATER© from all sampled fish for subsequent RNA extraction. For the cohort that was challenged 120 days post vaccination, an additional sampling was done 7 weeks post challenge. In addition to heart and kidney for RNALATER®, hearts (ventricle and atrium) were also fixed in formalin from all fish on this last sampling point (week 7) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 3 according to severity, where 0 indicates no pathologic finding and 3 indicate presence of severe pathology (service provided by PHARMAQAnalytiq, Norway).

The efficacy of the vaccines was evaluated by challenge after up to 26 weeks of immunization (challenge at 12, 18- and 26-weeks post immunization). The challenge at week 18 was followed for 50 days with two sampling points, at 20- and 50-days post challenge, where heart and kidney tissues were sampled for analysis by qPCR and evaluation of histopathology in heart as described above.

The results showed that significant protection against PMCV infection was achieved with all vaccines until the last challenge, 183 days/2196 degree days post vaccination. The combination of G-ORF1 and IFNb provided better protection than the two components separately (Tables 23, 24). The overall results show that the vaccine provide long and durable protection.

TABLE 23
Tank 1. Viral RNA in heart 3 weeks post challenge with three
different durations of immunization before challenge. Mean Ct-value for
PMCV with standard deviation is indicated for each group.
N = 15 per group, per sampling-point.

| Vaccine- | | 12 weeks | 18 weeks | 26 weeks |
|---|---|---|---|---|
| group | Description | immunization | immunization | immunization |
| 6 | PBS | 27.42 ± 2.07 | 29.33 ± 1.62 | 29.33 ± 1.74 |
| 2 | G-ORF1 | 28.70 ± 2.19 | 30.50 ± 2.38 | 29.62 ± 3.59 |
| 3 | IFNb | 33.82 ± 3.48 | 32.65 ± 3.68 | 28.35 ± 6.22 |
| 1 | G-ORF1 + IFNb | 35.88 ± 3.25 | 33.25 ± 3.36 | 32.21 ± 1.95 |

TABLE 24
Tank 2. Viral RNA in heart 3 weeks post challenge with three different
durations of immunization before challenge. Mean Ct-value for PMCV
with standard deviation is indicated for each group. N = 15
per group, per sampling-point.

| Vaccine- | | 12 weeks | 18 weeks | 26 weeks |
|---|---|---|---|---|
| group | Description | immunization | immunization | immunization |
| 6 | PBS | 27.68 ± 2.32 | 27.58 ± 2.04 | 28.80 ± 1.90 |
| 4 | G-ORF1-CMV- | 36.73 ± 4.69 | 33.75 ± 2.80 | 32.93 ± 3.31 |
| | IFNb | | | |
| 5 | G-ORF1-CMV- | 33.58 ± 3.10 | 30.70 ± 2.14 | 32.63 ± 4.13 |
| | IFNb | | | |

TABLE 25
Tank 1. Histopathological lesions (histoscore) in atrium 7 weeks
post challenge after an immunization period of 18 weeks. The indicated
p-value was calculated by comparing individual groups with
the PBS control group using a One-way ANOVA, with Dunnett's
post test. NA = Not applicable.

| | | Mean | Standard | | p-value |
|---|---|---|---|---|---|
| Vaccine- | | histoscore, | deviation, | | (compared to |
| group | Description | Atrium | histoscore | N | PBS) |
| 6 | PBS | 1.54 | 1.08 | 12 | NA |
| 2 | G-ORF1 | 0.60 | 0.74 | 15 | 0.01 |
| 3 | IFNb | 0.43 | 0.78 | 15 | 0.0025 |
| 1 | G-ORF1 + IFNb | 0.43 | 0.65 | 14 | 0.0028 |

TABLE 26
Tank 2. Histopathological lesions (histoscore) in atrium 7 weeks post
challenge after an immunization period of 18 weeks. The indicated
p-value was calculated by comparing individual groups with the PBS
control group using a One-way ANOVA, with Dunnet's post test.
NA = Not applicable.

| | | Mean | Standard | | p-value |
|---|---|---|---|---|---|
| Vaccine- | | histoscore, | deviation, | | (compared |
| group | Description | Atrium | histoscore | N | to PBS) |
| 6 | PBS | 1.68 | 0.75 | 11 | NA |
| 4 | G-ORF1-CMV-IFNb | 0.07 | 0.18 | 15 | <0.0001 |
| 5 | G-ORF1-CMV-IFNb | 0.50 | 0.76 | 8 | 0.0002 |

### Example 7—Optimization of Adjuvant Efficiency

In an attempt to further optimize the adjuvant potential of IFNb, the inventors identified homologous genes in Atlantic salmon and closely related species. As salmonids have experienced an evolutionary recent whole genome duplication, many salmon genes are found in duplicates (diverged homologues). Three new genes, similar but not identical to the initially tested IFNb-adjuvant were selected (Table 27). These were:
Atlantic salmon (Salmo salar)—IFNb1 (diverged paralog gene)
Brown trout (Salmo trutta)—IFN a3-like gene
Sockeye salmon (Onchorhynchus nerka)—IFNa3-like gene.

TABLE 27

| | GenBank | | | | | |
|---|---|---|---|---|---|---|
| Gene | accession # | | 1 | 2 | 3 | 4 |
| IFNb Salmo salar (SEQ | | 1 | | 6 | 5 | 11 |
| ID NO: 6) | | | | | | |
| IFNb1 Salmo salar | EU768890.1 | 2 | 96.74 | | 9 | 13 |
| IFNa3 like Salmo trutta | XM_029727364.1 | 3 | 97.28 | 95.11 | | 11 |
| IFNa3 like | XM_029635101.1 | 4 | 94.02 | 92.93 | 94.02 | |
| Onchorhynchus nerka | | | | | | |

Pairwise comparison of alterative IFNb adjuvants showed as amino-acid identity (%) and number of different amino acids (italicized)

As vaccine integration into the salmon genome through homologous recombination might be a concern, risk mitigation can be obtained by reducing the sequence similarity between the adjuvant and the salmon genome. This was done by first translating the plasmid encoded adjuvants to protein sequences. Thereafter these sequences were reverse translated from proteins back into nucleotide sequences (back-translation) based on codon frequency distribution (codons assigned with a probability given by the frequency of use in Atlantic salmon). Reverse translation was done using CLC Main Workbench (Qiagen) with software settings to use codons based on frequency distribution.

The new adjuvant sequences returned after this procedure were between 74-80% identical to the wild type IFNb sequences at the nucleotide level. The activities of the four original native adjuvant candidates, including the four codon-changed versions (referred to as RT) were then compared using an in vitro assay.

Thus, the wild type nucleic acid sequence for IFNb (SEQ ID NO: 18) was about 76% identical to the RT nucleic acid sequence for IFNb (SEQ ID NO: 17), and the wild type nucleic acid sequence for IFNb1 (SEQ ID NO: 19) was about 78% identical to the RT nucleic acid sequence for IFNb1 (SEQ ID NO: 20)

To facilitate testing of the different IFNb candidates and assess their adjuvant potential, an in vitro screening assay was established. The principle of this test is to quantify Mx-expression (IFN-induced gene) by RT-qPCR. Mx proteins belong to the superfamily of large GTPases with antiviral activity against a wide range of RNA viruses. In vivo, the expression of Mx genes is tightly regulated by the presence of type I interferons (IFNs), and their induction has been described during several viral infections. As CHH-1 cells do not express Mx in response to IFNb stimulation (the potential result of lacking receptors) and TO cells not easily can be transfected, the assay has been set up in the following way:

Functionality/activity of IFNb genes was studied by transfecting CHH-1 cells with the various adjuvant constructs with non-adjuvanted constructs included as negative controls. Seven days post transfection, cell culture medium containing IFN molecules secreted from the CHH-1 cell transfectants was collected and transferred to another fish cell-line (TO cells) in different dilutions. The final ratio of conditioned medium used for incubation of TO cells was typically 1:6; 1:60, 1:600 and 1:6000, allowing the in vitro model also to provide quantitative measures of adjuvant activity. Following three days of incubation with conditioned medium, TO cells were sampled by carefully removing the cell culture medium and extracting RNA from the cells. The Mx response induced by IFNb was analyzed by qPCR using the following primers/probe:

SEQ ID NO: 42 Mx Fwd:
GATGCTGCACCTCAAGTCCTATTA
SEQ ID NO: 43 Mx Rev:
CGGATCACCATGGGAATCTGA
SEQ ID NO: 44 Mx Probe:
6-FAM-CAGGATATCCAGTCAACGTT-MGB

The results are provided in Table 28 below:

TABLE 28

| | Ratio CHH-1 conditioned | | |
|---|---|---|---|
| | medium transferred to | | |
| | TO-cells | | |
| Construct | 1:6 | 1:60 | 1:600 |
| Salmo Salar IFNb wild type (SEQ ID NO: 18 | 21.61 | 24.29 | 34.44 |
| Salmo Salar IFNb RT (SEQ ID NO: 17) | 20.86 | 22.33 | 26.39 |
| Salmo Salar IFNb1 wild type (SEQ ID NO: 19) | 20.94 | 21.77 | 23.66 |
| Salmo Salar IFNb1 RT (SEQ ID NO: 20) | 20.57 | 21.05 | 21.84 |
| Salmo trutta IFNa3 like wild type | 20.66 | 22.17 | 27.04 |
| Salmo trutta IFNa3 like RT | 21.49 | 21.96 | 26.82 |
| Onchorhynchus nerka INFa3 wild type | 20.99 | 24.04 | 31.26 |
| Onchorhynchus nerka INFa3 RT | 21.28 | 22.87 | 28.13 |
| eGFP | No Ct | No Ct | No Ct |
| Non template control | No Ct | | |

Real-time Ct values for Mx in TO cells incubated with different concentrations of conditioned medium from transfected CHH-1 cells. Lower Ct values indicate higher levels of IFN induction.

These results demonstrate that RT constructs have slightly higher immunomodulatory activity compared to the wild type sequences. The results also clearly show that IFNb1 possesses higher immunomodulatory activity than IFNb.

Based on the in vitro studies, the four RT INFb adjuvants were selected for testing as vaccine adjuvants in fish (all groups receiving the PMCV capsid antigen (G-ORF1) and adjuvant on two separate plasmids). In addition, one group received G-ORF1 vaccine antigen plus an eGFP encoding plasmid as negative adjuvant control group. All fish received one intramuscular injection on each side of the fish. All fish received the antigen and the plasmid encoded adjuvant on separate plasmids, the dose for each plasmid was 20 g. A non-immunized control group was included and received 2×50 μl PBS, as summarized in Table 29.

TABLE 29

| | | Detailed | | | | | Final | Dose μg |
|---|---|---|---|---|---|---|---|---|
| | Description | description | Antigen | Description | Adjvuant | Final conc | conc B | (2 × 0.05 |
| Group | (Antigen) | Antigen | backbone | (Adjuvant) | backbone | A μg/ml | (μg/ml) | ml) |
| 1 | G-ORF1 | G- | NTC9385R | IFNb S. | pVAX1 | 200 | 200 | 20 each |
| | | PMCV_ORF | | salar | | | | |
| | | 1_full_1- | | | | | | |
| | | 2583 | | | | | | |
| 2 | G-ORF1 | G- | NTC9385R | IFNb1_S. | pVAX1 | 200 | 200 | 20 each |
| | | PMCV_ORF | | salar | | | | |
| | | 1_full_1- | | | | | | |
| | | 2583 | | | | | | |
| 3 | G-ORF1 | G- | NTC9385R | IFNb_S. | pVAX1 | 200 | 200 | 20 each |
| | | PMCV_ORF | | trutta | | | | |
| | | 1_full_1- | | IFNa3-like | | | | |
| | | 2583 | | | | | | |
| 4 | G-ORF1 | G- | NTC9385R | IFNb_O. | pVAX1 | 200 | 200 | 20 each |
| | | PMCV_ORF | | nerka INFa3- | | | | |
| | | 1_full_1- | | like | | | | |
| | | 2583 | | | | | | |
| 5 | G-ORF1 | G- | NTC9385R | eGFP | pcDNA3.1(+) | 200 | 200 | 20 each |
| | | PMCV_ORF | | | | | | |
| | | 1_full_1- | | | | | | |
| | | 2583 | | | | | | |
| 6 | PBS | PBS control | NA | | | | | |

Atlantic salmon (Salmo salar) (n=30 per group) kept in freshwater and with average weight of 20 grams were anaesthetized using Tricain (PHARMAQ), tagged by shortening of the adipose fin and/or maxillae, allocated to a group, and intramuscularly injected twice under the same anaesthetic period (one injection on each side of the fish) with 0.05 ml of the test vaccines. Immunity was allowed to develop for 48 days at 12° C. (light:dark 12:12) before the fish were anaesthetized again using Tricain (PHARMAQ) and challenged with infectious PMCV by intraperitoneal injection of 0.1 ml of homogenized heart (isolate ID ALV1273 Heart) originating from a Norwegian field outbreak of CMS. The fish (n=15 per group) were then sampled at two timepoints; 19 days and 49 days post challenge. Heart ventricle and kidney were collected on RNALATER® from all sampled fish for subsequent RNA extraction. Hearts (ventricle and atrium) were also fixed in formalin from all fish on the last sampling point (day 49) for histopathological analysis. RNA was extracted from all samples stored on RNALATER® and analyzed by Real-Time RT-PCR for presence of PMCV RNA using a commercially available service from PHARMAQ Analytiq (Norway). Hearts stored on formalin were sectioned and histopathology was scored from 0 to 3 according to severity, where 0 indicates no pathologic finding and 3 indicate presence of severe pathology (service provided by PHARMAQ Analytiq, Norway).

Results are shown in tables 30 and 31 below.

TABLE 30
Tank 1. Viral RNA in kidney 3 weeks post challenge. The indicated
p-value was calculated by comparing individual groups with the PBS
control group using a One-way ANOVA, with Dunnett's post test.
NA = Not applicable.

| | | Mean Ct- | Standard | | p-value |
|---|---|---|---|---|---|
| Vaccine | | value, | deviation, | | (compared |
| group | Description | PMCV | Ct | n | to PBS) |
| 6 | PBS | 19.89 | 0.79 | 15 | NA |
| 1 | G-ORF1 + IFNb | 25.42 | 1.51 | 15 | <0.0001 |
| | S. salar | | | | |
| 2 | G-ORF1 + IFNb1 | 27.66 | 1.31 | 15 | <0.0001 |
| | S. salar | | | | |
| 3 | G-ORF1 + IFNb a3 | 27.13 | 1.09 | 15 | <0.0001 |
| | S. trutta | | | | |
| 4 | G-ORF1 + IFNb a3 | 26.74 | 1.25 | 15 | <0.0001 |
| | O. nerka | | | | |
| 5 | G-ORF1 + EGFP | 23.23 | 2.10 | 15 | 0.028 |

TABLE 31
Tank 2. Viral RNA in kidney 3 weeks post challenge. The indicated p-value
was calculated by comparing individual groups with the PBS control
group using a One-way ANOVA, with Dunnett's post test.
NA = Not applicable.

| | | Mean Ct- | Standard | | p-value |
|---|---|---|---|---|---|
| Vaccine | | value, | deviation, | | (compared |
| group | Description | PMCV | Ct | n | to PBS) |
| 6 | PBS | 20.73 | 1.44 | 15 | NA |
| 1 | G-ORF1 + IFNb | 26.55 | 0.94 | 15 | <0.0001 |
| | S. salar | | | | |
| 2 | G-ORF1 + IFNb1 | 26.27 | 0.99 | 15 | <0.0001 |
| | S. salar | | | | |
| 3 | G-ORF1 + IFNb a3 | 26.72 | 1.15 | 15 | <0.0001 |
| | S. trutta | | | | |
| 4 | G-ORF1 + IFNb a3 | 26.43 | 1.09 | 11 | <0.0001 |
| | O. nerka | | | | |
| 5 | G-ORF1 + EGFP | 21.63 | 1.76 | 15 | 0.20 |

The overall conclusion is that all tested RT adjuvants tested provide similar protection, although the protection elicited using IFNb1 RT construct was slightly higher than then protection elicited by IFNb RT construct. Codon-changed versions of these adjuvants are preferred to reduce risk of homologous recombination.

The best way to deliver the adjuvant is on the same plasmid as the antigen while using a separate but identical promoter as the antigen but delivery of the antigen and the adjuvant using different plasmids is also effective.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A fusion protein comprising, from N-terminus to C-terminus:
a) N-terminal secretion signal sequence of a secreted or a first membrane-bound protein;
b) an antigen from a non-enveloped virus;
c) a transmembrane domain of a second membrane-bound protein.

2. The fusion protein of claim 1, wherein said first protein is selected from the group consisting of Intereferon a, Interferon b, Interferon c, Interferon d, Interferon gamma, interleukine-2, interleukine 4, interleukine-12 Hemagglutinin (HE) protein of Infectious salmon anemia virus (ISAV), viral hemorrhagic septicemia virus G-protein and the fusion protein of Atlantic Salmon Paramyxovirus, and said second protein is independently selected from the group consisting of Hemagglutinin (HE) protein of Infectious salmon anemia virus (ISAV), viral hemorrhagic septicemia virus G-protein and the fusion protein of Atlantic Salmon Paramyxovirus

3. The fusion protein of claim 1 wherein the first membrane-bound protein is identical to the second membrane-bound protein.

4. The fusion protein of claim 3, wherein said membrane bound protein is viral hemorrhagic septicemia virus G-protein (VHSV-G).

5. The fusion protein of claim 4, wherein said N-terminal secretion signal sequence is at least 90% identical to SEQ ID NO: 7 or 48, or wherein said secretion signal sequence comprises a portion of SEQ ID NO: 48 or a sequence at least 90% identical to SEQ ID NO: 48, wherein said portion comprises SEQ ID NO: 7 or a sequence that is at least 90% identical to SEQ ID NO: 7.

6. The fusion protein of claim 5 wherein at least half of differing amino acids in said N-terminal secretion signal are conservative substitutions.

7. The fusion protein of claim 1, wherein said transmembrane domain is at least 90% identical to SEQ ID NO: 8 or 49, or wherein said transmembrane fragment comprises a portion of SEQ ID NO: 49 or a sequence at least 90% identical to SEQ ID NO: 49, wherein said portion comprises SEQ ID NO: 8 or a sequence that is at least 90% identical to SEQ ID NO: 8.

8. The fusion protein of claim 7 wherein at least half of differing amino acids in said Transmembrane domain are conservative substitutions.

9. The fusion protein of claim 1, wherein the non-enveloped virus is selected from the group consisting of Piscine Orthoreovirus (PRV), Piscine Myocarditis Syndrome Virus (PMCV), Infectious Pancreatic Necrosis Virus (IPNV), and betanodavirus.

10. The fusion protein of claim 9, wherein the antigen is PMCV ORF-1 antigen.

11.-44. (canceled)

45. A nucleic acid sequence that encodes the fusion protein of claim 1.

46. The nucleic acid sequence according to claim 45, comprising SEQ ID NO: 16.

47.-57. (canceled)

58. A vector comprising the nucleic acid sequence of claim 45.

59. The vector of claim 57 which is a plasmid vector.

60. A host cell comprising the vector of claim 58.

61. A vaccine comprising the vector of claim 58.

62. The vaccine according to claim 61 wherein the antigen is PMCV ORF-1 antigen.

63. A method of preventing piscine myocarditis virus (PMCV) infection in a salmonid in need thereof comprising administering to said salmonid the vaccine according to claim 62.

64. The method of claim 63, wherein said salmonid weighs between 15 and 200 grams.

65. The method of claim 63, wherein said salmonid weighs between 40 and 110 grams.

66. The method of claim 63, wherein said salmonid is Atlantic salmon (Salmo salar), rainbow trout (Oncorhynchus mykiss), or Coho Salmon (Oncorhynchus kisutch).

67.-74. (canceled)
